(19) United States
(12) Patent Application Publication
(10) Pub. No.: US 2025/0250535 A1
(43) Pub. Date: Aug. 7, 2025

(54) METHODS TO PRODUCE ACETYLATED AND NON-ACETYLATED GLYCOLIPID AMPHIPHILES

(71) Applicant: Universiteit Gent, Gent (BE)

(72) Inventors: Bart DEVREESE, Vosselare (BE); Zhoujian DIAO, Gent (BE); Wim SOETAERT, Lievegem (BE); Sophie ROELANTS, Melle (BE); Sofie DE MAESENEIRE, Destelbergen (BE); Goedele LUYTEN, Gent (BE); Sven DIERICKX, Gent (BE); Karolien MAES, Berlare (BE)

(73) Assignee: Universiteit Gent, Gent (BE)

(21) Appl. No.: 18/855,098

(22) PCT Filed: Apr. 27, 2023
(86) PCT No.: PCT/EP2023/061136

§ 371 Date: Oct. 8, 2024

(30) Foreign Application Priority Data
May 3, 2022 (EP) 22171389.4

(51) Int. Cl. C12N 1/16; C12N 9/10; C12N 9/24; C12P 19/44; C12R 1/72

(52) U.S. Cl. CPC C12N 1/16; C12N 9/1029; C12N 9/1051; C12N 9/2402; C12P 19/44; C12R 2001/72; C12Y 204/01017; C12Y 302/01107

(57) ABSTRACT

The present invention relates to the use of a known enzyme ‘denominated as a Starmerella bombicola lactone esterase (Sble)’ to perform a transesterification and/or hydrolysis reaction. More specifically the Sble enzyme performs a transesterification and/or hydrolysis reaction on bola amphiphilic glycolipids. The invention indeed discloses that said Sble is capable to convert bola sophorolipids into lactonic and/or acidic sophorolipids and saccharides, and, that yeast strains containing a non-functional or dysfunctional Sble enzyme and/or a disabled sble gene and/or not containing the sble gene produce (acetylated) bola amphiphilic glycolipids. In addition, the invention further discloses a method to produce non-acetylated (bola) amphiphilic glycolipids via rendering acetyltransferase enzymes At1, At2 and At3 non-functional or dysfunctional in the latter yeast strains and/or by modifying strains so that they do not contain the acetyltransferase αt1, αt2 and αt3 gene(s) and/or by strains not containing the αt1, αt2 and αt3 gene(s). Moreover, upon rendering the glucosyltransferase B (UgtB1) non-functional or dysfunctional in the abovementioned strains and/or upon removing and/or disabling the ugtB1 gene and/or by strains not containing the ugtB1 gene these produce acetylated and/or non-acetylated bola amphiphilic glucolipids. The invention further discloses a method to produce non-acetylated glycolipids via rendering acetyltransferase enzymes At1, At2 and/or At3 non-functional or dysfunctional and/or removing and/or disabling the glycolipid acetyltransferase genes in glycolipid producing yeast strains and/or by strains not containing the αt1, αt2 and αt3 gene(s).

### TECHNICAL FIELD OF THE INVENTION

The present invention relates to the use of a known enzyme ‘denominated as a Starmerella bombicola lactone esterase (Sble)’ to perform a transesterification and/or hydrolysis reaction. More specifically the Sble enzyme performs a transesterification and/or hydrolysis reaction on bola amphiphilic glycolipids. The invention indeed discloses that said Sble is capable to convert bola sophorolipids into lactonic (transesterification) and/or acidic sophorolipids and saccharides (hydrolysis), and, that yeast strains containing a non-functional or dysfunctional Sble enzyme and/or a disabled sble gene and/or have the sble gene removed produce (acetylated) bola amphiphilic glycolipids. In addition, the invention further discloses a method to produce non-acetylated (bola) amphiphilic glycolipids via rendering acetyltransferase enzymes At1, At2 and At3 non-functional or dysfunctional in the latter yeast strains and/or by modifying strains so that their acetyltransferase at1, at2 and at3 gene(s) are disabled and/or removed. Moreover, upon rendering the glucosyltransferase B (UgtB1) non-functional or dysfunctional in the abovementioned strains and/or upon removing and/or disabling the ugtB1 gene, these produce acetylated and/or non-acetylated bola amphiphilic glucolipids. The invention further discloses a method to produce non-acetylated glycolipids via rendering acetyltransferase enzymes At1, At2 and At3 non-functional or dysfunctional and/or removing and/or disabling the glycolipid acetyltransferase genes in glycolipid producing yeast strains.

### BACKGROUND ART

The yeast S. bombicola is, among others, well-known in the art for its high production of sophorolipids (SLs). SLs consist of the disaccharide sophorose linked to a hydroxylated fatty acid. The wild type S. bombicola strain produces a mixture of acidic and lactonic SLs, which can be non-, mono- or di-acetylated and mainly contain a C18:1 fatty acid. The SL biosynthetic pathway was previously elucidated, and the proposed pathway is shown in FIG. 1.

All but one gene involved in the SL biosynthesis are found in one large subtelomeric gene cluster (see FIG. 1). The current understanding of the SL biosynthetic pathway involves five steps with di-acetylated lactonic SLs as the final product: The first step consists of (sub) terminal hydroxylation of a fatty acid by the action of a cytochrome P450 monooxygenase (Cyp52M1) (Van Bogaert et al., 2009a) (FIG. 1 step/gene (1)). Subsequent glycosylation of the hydroxy fatty acid involves two glucosyltransferases. The first one (Ugta1) (Saerens et al., 2011a) is responsible for the transfer of a glucose molecule from UDP-glucose to the hydroxylated fatty acid yielding a glucolipid and UDP (FIG. 1 step/gene (2)) while the second one (Ugtb1) (Saerens et al., 2011c) specifically transfers a second glucose molecule from UDP glucose to the formed glucolipid (and not to the hydroxylated fatty acid) (FIG. 1 step/gene 3)). The SLs are subsequently acetylated by the action of an acetyltransferase (At1) (Saerens et al., 2011b) (FIG. 1 step/gene 4 and can be further lactonised by the action of a secreted lactone esterase (Sble) (FIG. 1 step/gene 6) (Ciesielska et al. 2014, WO 2013/092421) after secretion by a specific SL transporter (Mdr) (FIG. 1 step/gene 5). In contrast to all the other genes involved in SL biosynthesis by S. bombicola, the sble gene is not located in the biosynthetic gene cluster and seems to be differently regulated. This last step is also (mainly) performed in the extracellular space as the Sble protein possesses a secretion signal and is actively secreted and abundantly found in the extracellular space (Ciesielska et al. 2014). An SL transporter (Mdr) is also encoded within the biosynthetic gene cluster. Deletion of this gene results in a decrease of at least 90% of SL production (Van Bogaert et al., 2013).

A single S. bombicola at1 deletion strain S. bombicola Δat1 has been described by Saerens et al., (2011b) and was described to produce non-acetylated acidic and non-acetylated lactonic sophorolipids (SLs). Non-acetylated lactonic SLs were reported to be the most predominant structures in the mixture, in addition to minor amounts of open-ring/acidic SLs. A single S. bombicola sble deletion strain S. bombicola Δsble was later described by Ciesielska et al. (2014) and reported to exclusively produce acidic SLs. The authors also suggested that the SLs are secreted by the yeast in the acidic form and are subsequently lactonized by the extracellularly secreted Sble enzyme. Roelants et al. (2016) also reported production experiments with this strain, which was again reported to exclusively produce acidic SLs in a mixture of acetylated and non-acetylated congeners. Ciesielska et al. (2016) later investigated the mode of action of the Sble enzyme through in vitro enzyme assays and reported it to catalyze the intramolecular esterification (lactonization) of acetylated acidic sophorolipids in an aqueous environment into acetylated lactonic sophorolipids. No lactonization activity was observed at all by the authors for non-acetylated acidic SLs, so acetylation was deemed to be essential for this esterification reaction to occur by Sble. The acetylated acidic SLs used in the in vitro Sble enzyme assays described in the abovementioned work were obtained from the Δsble S. bombicola strain (Ciesielska et al. 2014) and HPLC and LC-MS analyses suggested that the extracted SLs used for these in vitro enzyme assays consisted of a mixture composed of 50% non-acetylated acidic SLs, 17% mono-acetylated acidic SLs, 31% di-acetylated acidic SLs and 2% contaminants as described by the authors in the art.

Subsequently, it was found that a combination of these two deletions in one strain (i.e. S. bombicola Δat1 Δsble) unexpectedly resulted in the biosynthesis of bolaform/bola amphiphilic glycolipids of which the general formula is shown in FIGS. 2A and 2B. More specifically, the authors reported the biosynthesis of non-acetylated bola sophorolipids (FIG. 2C) (Van Bogaert et al., 2016 and WO2015/028278), which was described also in subsequent works (Van Renterghem et al., 2019, WO/2021/229017). Based on the previous findings and the proposed biosynthetic pathway shown in FIG. 1, this strain should logically produce non-acetylated acidic sophorolipids. Surprisingly, in addition to these anticipated non-acetylated acidic SLs, also bola sophorolipids (74% of the produced SLs) were obtained. These bola sophorolipids contain an additional sophorose molecule linked to the carboxyl function of the acidic sophorolipids as confirmed by LC-MS and NMR analysis. Bola sophorolipid biosynthesis was proven to be attributed to the promiscuous activity of both UDP-glucosyltransferases UgtA1 and UgtB1 from the sophorolipid biosynthetic pathway, found to also display activity towards the carboxyl group of non-acetylated intermediates. The absence of acetyl groups was hypothesized to trigger formation of bola glycolipid compounds starting from acidic sophorolipids as they were found to be produced by the Δat1 Δsble strain and not by the Δsble strain. The authors also hinted this as the potential reason why these bola sophorolipids are only detected in marginal amounts (<0.1% of produced sophorolipids) in cultures of the wild type Starmerella bombicola strain (Price at al., 2012) as the presence of the At1 enzyme in the wild type strain is giving rise to acetylated sophorolipids, which would thus hamper the formation of bolaform sophorolipids. LC-MS analysis of the produced sophorolipid mixture by the Δat1 Δsble strain revealed the production of non-acetylated bola sophorolipids with variation in the incorporated fatty acid chain length and position of the hydroxyl group on the fatty acid (as is also the case for wild type sophorolipids). Fractionation on the sophorolipid mixture produced by this new strain was performed and NMR analysis confirmed the structure of non-acetylated bola sophorolipids as shown in FIG. 2. Because this was an unexpected finding, the authors again investigated the glycolipid mixtures produced by both the single deletion strains described above. The Δsble S. bombicola strain was confirmed to only produce acidic sophorolipids by the authors (Van Bogaert et al., 2016), while the Δat1 S. bombicola strain was, upon reanalysis using an adapted glycolipid extraction protocol for more hydrophilic compounds, found to also produce non-acetylated bola sophorolipids in addition to the previously reported non-acetylated acidic and lactonic sophorolipids reported to be produced by this strain (Van Bogaert et al., 2016). The authors thus suggested again—as mentioned above—that the absence of acetylation seems to be a key factor triggering bola sophorolipid synthesis and suggested that this effect is enhanced by the absence of lactonic sophorolipid forms (where the carboxyl group is not freely available anymore) as better production efficiencies of bola sophorolipids seem to be obtained with the double deletion strain. Non-acetylated glycolipid compounds were hypothesized to allow a certain conformational orientation in the UgtA1 and UgtB1 enzymes, which would not be possible for the acetylated equivalents, thus resulting in further glycosylation of non-acetylated acidic sophorolipids resulting in non-acetylated bola sophorolipids.

It is however currently unknown whether yeasts strains which comprise a non-functional or dysfunctional lactone esterase enzyme (Sble) and a functional acetyltransferase enzyme 1 (At1) are capable to produce bola sophorolipids. It is also unknown that acetylated bola sophorolipids can be produced. It is also unknown whether yeast strains which comprise a non-functional or dysfunctional acetyltransferase enzyme (At1), encoded in the SL biosynthetic gene cluster, and responsible for sophorolipid acetylation, are still capable to produce acetylated (bola) sophorolipids. Moreover, it is completely unknown whether the Sble enzyme is capable to perform a transesterification reaction, more specifically it is completely unknown that the Sble enzyme has transesterification activity on bola sophorolipids/bola glucolipids giving rise to into lactonic sophorolipids/glucolipids respectively. It is also unknown that the Sble enzyme has hydrolysis activity on bola sophorolipids/glucolipids giving rise to acidic sophorolipid/glucolipids.

### BRIEF DESCRIPTION OF FIGURES

FIG. 1: (a) Illustration of chromosome II of S. bombicola containing the sophorolipid biosynthetic gene cluster (±11 kb) and the gene responsible for lactonisation (sble) at the other side of the chromosome (b) The full sophorolipid biosynthetic pathway consisting of (1) hydroxylation of a fatty acid (mainly C16 or C18) by a Cyp52M1 monooxygenase (2) glucosylation of the FA-OH by the first glucosyltransferase UgtA1 (3) and second glucosylation step of the formed glucolipid by a second glucosyltransferase UgtB1 giving rise to an acidic sophorolipid, which can be (4) acetylated by the action of an acetyltransferase At1. The different sophorolipids are transported into the extracellular space by a multidrug transporter protein (Mdr) (5). Lactonisation (6) mainly occurs extracellularly as the responsible enzyme (Sble) is secreted.

FIG. 2: (A and B) Bolaform/bola amphiphilic glycolipids R₁=H or CO—CH₃; R₂=H or CO—CH₃; R₃=H or CO—CH₃; R₄=H or CO—CH₃; R₅=an unsubstituted or hydroxy-substituted, unbranched, optionally one to three double or triple bonds containing, divalent organic moiety comprising 6 to 32 carbon atoms, R₆=H, CH₃ or an unsubstituted or hydroxy-substituted, unbranched, optionally one to three double or triple bonds containing, organic moiety comprising 2 to 10 carbon atoms; n=1 or 0; m=1 or 0. (C) Non-acetylated bola sophorolipids as reported to be produced by the S. bombicola Δat1 Δsble strain and also by the S. bombicola Δat1 strain. Only non-acetylated variants were reported to be produced by these strains by Van Bogaert et al., 2016.

FIG. 3: Deletion cassette 1 for the complete deletion of the sble gene in S. bombicola with an ura3 marker. HR: homologous region.

FIG. 4: Deletion cassette 2 for the deletion of the at1 gene in S. bombicola with an ura3 marker. HR: homologous region.

FIG. 5: Deletion cassette 3 for the deletion of the at1 gene in S. bombicola with a hygromycine resistance (HygroR) marker. HR: homologous region

FIG. 6: Acetylated bola sophorolipid with an acetylation degree of 4 i.e. acetylgroups are present on positions 6′ and 6″ of all incorporated glucose moieties.

FIG. 7: HPLC-UV chromatograms of samples from of the activity assay of rSble (1) with acetylated acidic sophorolipids with terminal hydroxylation (attachment of sophorose at the terminal position of the fatty acid, code), after 1 h incubation at 30° C., 1400 rpm and pH3.5. rSble was added at a concentration of 4 ug/ml, acetylated acidic sophorolipids at a concentration of 5 mM. The black arrow indicates the main substrate. (2) negative control (without addition of enzyme)

FIG. 8: HPLC-UV chromatograms of samples from the activity assay of rSble (1) with acetylated acidic sophorolipids with attachment of the glycosyl group mainly at the subterminal position, after 1 h incubation at 30° C., 1400 rpm and pH3.5. rSble was added at a concentration of 4 ug/ml, acetylated acidic sophorolipids at a concentration of 5 mM. The black arrow indicates the main substrate (2) negative control of this experiment (without addition of enzyme)

FIG. 9: HPLC-UV chromatograms of samples from the activity assay of rSble (1) with non-acetylated acidic sophorolipids with attachment of glycosyl group mainly at subterminal position after 1 h incubation at 30° C., 1400 rpm and pH3.5. rSble was added at a concentration of 4 ug/ml, non-acetylated acidic sophorolipids at a concentration of 5 mM. The black arrow indicates the main substrate (2) negative control of this sample (without addition of enzyme)

FIG. 10: HPLC-UV chromatograms of samples from (1) the negative control of an sophorolipid mixture obtained from the Δsble strain used for the activity assay (2) the activity assay of rSble using the acidic sophorolipid mixture shown in panel 1 as substrate. The five-angle stars in panel 2 indicate the produced lactonic sophorolipids after incubation. The arrows in panel 1 indicate the three peaks with significant decrease after reaction with the addition of the rSble enzyme (shown in panel 2), black four-angle stars (in panel 1 and 2) indicate the peak corresponding to di-acetylated acidic sophorolipids (C18:1) with subterminal (ω-1) attachment, of which no reduction is observed upon addition of rSble.

FIG. 11: LC-MS TIC chromatograms from an activity assay of rSble containing mainly acetylated bola sophorolipids (code: INV-113). The top chromatogram is the mixture to which no enzyme is added. Peaks indicated with an arrow are all mono- and di-acetylated bola sophorolipids (see Table 5) that decreased in intensity after incubation with the enzyme. The lower chromatogram is obtained from the same sophorolipid mixture, but is obtained after incubation with rSble. All indicated peaks are lactonic sophorolipids (see Table 5).

FIG. 12: MS TIC chromatograms from an activity assay of rSble containing mainly non-acetylated bola sophorolipids (code: INV-22). The top chromatogram is the mixture to which no enzyme is added. Peaks indicated with an arrow are mainly non-bola sophorolipids (see Table 6) that decreased in intensity after incubation with the enzyme. The lower chromatogram is obtained from the same sophorolipid mixture, but is obtained after incubation with rSble. Indicated peaks correspond to non- and mono-acetylated lactonic sophorolipids (see Table 6).

FIG. 13. Adapted sophorolipid biosynthesis pathway in S. bombicola. (1) Cyp52M1, (2) UgtA1, (3) UgtB1, (4) At1, (5) Sble. UDP: uridine diphosphate, CoA: coenzyme A, nA: non-acetylated, DiAC: di-acetylated, tetraAc: tetra-acetylated. In the figure the formation of tetra-acetylated bola sophorolipids and conversion thereof into di-acetylated lactonic sophorolipid and release of acetylated sophorose is shown. Bola sophorolipids with a lower acetylation degree are also converted into non- and mono-acetylated lactonic sophorolipids and non- and/or mono-acetylated sophorose.

FIG. 14. Deletion cassette for the deletion of the at2 gene in S. bombicola with the URA3 marker. HR: homologous region.

FIG. 15. Deletion cassette for the deletion of the at3 gene in S. bombicola with the URA3 marker. HR: homologous region. The 5′ homologous region coincides with the last 500 basepairs of the at3 coding sequence, which is indicated by the hatched bar.

### SUMMARY OF INVENTION

The present invention relates to:

The usage of a modified yeast strain which comprises a non-functional or dysfunctional transesterification enzyme Sble, and/or does not comprise a functional sble gene and/or has a reduced expression of sble compared to a non-modified yeast to produce bola amphiphilic glycolipids.

The usage of a modified yeast strain as described above wherein said bola amphiphilic glycolipids are acetylated bola amphiphilic glycolipids.

The usage of a modified yeast strain as described above wherein said bola amphiphilic glycolipids are bola sophorolipids.

The usage of a modified yeast strain as described above wherein said acetylated bola sophorolipids have an acetylation degree of 4.

The usage of a modified yeast strain as described above which further comprises a non-functional or dysfunctional glucosyltransferase enzyme UgtB1, and/or does not comprise a functional ugtB1 gene and/or has a reduced expression of ugtB1 compared to a non-modified yeast and wherein said bola amphiphilic glycolipids are bola glucolipids.

The usage of a modified yeast strain as describe above which further comprises a non-functional or dysfunctional acetyl transferase enzyme (At1), and/or does not comprise a functional at1 gene from the SL biosynthetic cluster and/or has a reduced expression of at1 compared to a non-modified yeast and wherein said acetylated bola amphiphilic glycolipids have an acetylation degree of 0, 1 or 2.

The usage of a modified yeast strain as described above which further comprises a second (At2) or a third (At3) non-functional or dysfunctional glycolipid acetylating enzyme, and/or does not comprise a functional at2 or at3 gene and/or has a reduced expression of at2 or at3 compared to a non-modified yeast wherein said acetylated bola amphiphilic glycolipids have an acetylation degree of 0, 1 or 2.

The usage of a modified yeast strain as described above which further comprises a second (At2) and a third (At3) non-functional or dysfunctional glycolipid acetylating enzyme, and/or does not comprise a functional at2 and at3 gene and/or has a reduced expression of at2 and at3 compared to a non-modified yeast to produce non-acetylated bola amphiphilic glycolipids, wherein said bola amphiphilic glycolipids are non-acetylated bola sophorolipids and/or non-acetylated bola glucolipids.

An isolated acetyltransferase having an amino acid sequence given by SEQ ID N° 6 or SEQ ID N° 8.

The usage of a modified yeast strain which comprises a non-functional or dysfunctional At1, At2 and At3 enzyme and/or not containing the at1, at2 and at3 genes and/or wherein the genes encoding for the At1, At2 and At3 enzymes are completely disabled or removed to produce non-acetylated glycolipids.

The usage of a modified yeast strain as described above according which comprises a non-functional or dysfunctional Sble, UgtB1, At1, At2 and/or At3 enzyme and/or not containing an sble, ugtB1, at1, at2 and/or at3 gene.

The usage of a modified yeast strain as described above wherein said yeast strain is a yeast strain selected from the strain selected of Starmerella (Candida) bombicola, Starmerella (Candida) apicola, Starmerella (Candida) batistae, Starmerella (Candida) magnolia, Candida gropengiesseri, Starmerella (Candida) floricola, Candida tropicalis, Candida riodocensis, Starmerella (Candida) stellata, Starmerella (Candida kuoi), Candida tropicalis, Candida sp. NRRL Y-27208, Pseudohyphozyma (Rhodotorula, Candida) bogoriensis sp., Wickerharmiella domericqiae, Candida antarctica, Pseudohyphozyma antarctica, Pseudohyphozyma bogoriensis, Candida lipolytica and a sophorolipid-producing strain of the Starmerella clade.

The usage of an Sble enzyme to perform a transesterification and/or hydrolysis reaction.

The usage of an Sble enzyme to perform a transesterification and/or hydrolysis reaction on bola amphiphilic glycolipids.

The usage of an Sble enzyme as described above to convert said bola amphiphilic glycolipids into lactonic glycolipids wherein said bola amphiphilic glycolipids are bola sophorolipids and wherein said lactonic glycolipids are lactonic sophorolipids, or, wherein said bola amphiphilic glycolipids are bola glucolipids and wherein said lactonic glycolipids are lactonic glucolipids and wherein glucose and/or sophorose which are non-acetylated and/or acetylated are released.

The usage of an Sble enzyme as described above to convert said bola amphiphilic glycolipids into acidic glycolipids wherein said bola amphiphilic glycolipids are bola sophorolipids and wherein said acidic glycolipids are acidic sophorolipids, or, wherein said bola amphiphilic glycolipids are bola glucolipids and wherein said acidic glycolipids are acidic glucolipids and wherein glucose and/or sophorose which are non-acetylated and/or acetylated are released.

The usage of an Sble enzyme as described above wherein said bola sophorolipids are tetra-acetylated bola sophorolipids and wherein said lactonic sophorolipids are di-acetylated lactonic sophorolipids and wherein glucose and/or sophorose which are non-acetylated and/or acetylated are released.

The usage of an Sble enzyme as described above wherein said bola sophorolipids are non, mono-, di- and/or tri-acetylated bola sophorolipids and wherein said lactonic sophorolipids are non-, mono- and/or di-acetylated lactonic sophorolipids and wherein glucose and/or sophorose which are non-acetylated and/or acetylated are released.

The usage of an Sble enzyme as described above wherein said bola sophorolipids are tetra-acetylated bola sophorolipids and wherein said acidic sophorolipid are di-acetylated acidic sophorolipids wherein glucose and/or sophorose which are non-acetylated and/or acetylated are released.

The usage of an Sble enzyme as described above wherein said bola sophorolipids are non, mono-, di- and/or tri-acetylated bola sophorolipids and wherein said acidic sophorolipids are non-, mono- and/or di-acetylated acidic sophorolipids and wherein glucose and/or sophorose which are non-acetylated and/or acetylated are released.

### DESCRIPTION OF INVENTION

The present invention relates to the surprising finding that the Sble enzyme is capable to perform a transesterification and/or a hydrolysis reaction on bola amphiphilic glycolipids. Such transesterification reaction refers to the process of the displacement of the alcohol from an ester by another one in a process similar to hydrolysis, but using an alcohol instead of water. Hydrolysis is thus the process of the displacement of the alcohol from an ester by water.

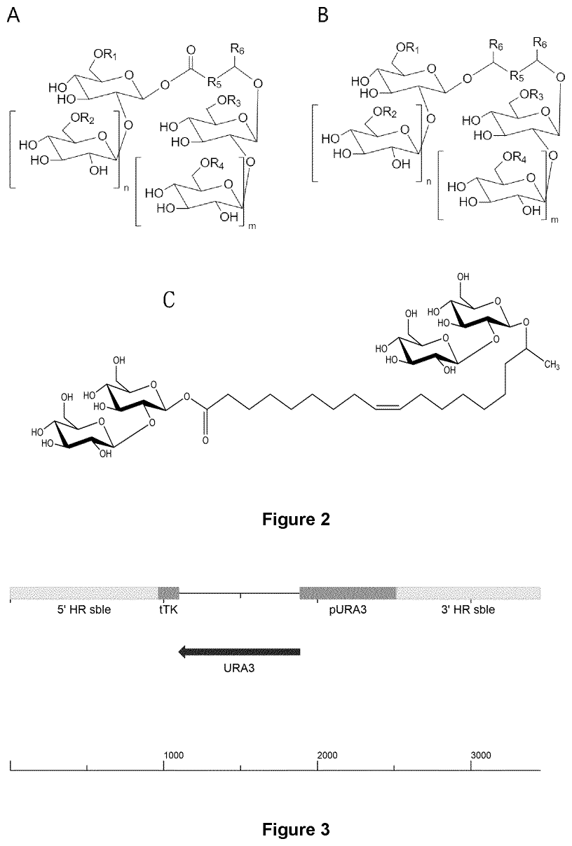

The term ‘bola amphiphilic glycolipids’ in the present invention refers to molecules as described by WO2015/028278 and are in general compounds with the general formula as shown in FIG. 2 (A and B). The present invention more specifically relates to the fact that the Sble enzyme is capable to perform a transesterification reaction on (acetylated) bola amphiphilic glycolipids. More specifically the Sble enzyme comprises transesterification activity on (acetylated) bola sophorolipids and bola glucolipids and converts these respectively into (acetylated) lactonic sophorolipids and lactonic glucolipids while releasing (acetylated) saccharides. Similar to transesterification, also hydrolysis of bola amphiphilic glycolipids is an activity found for the Sble enzyme: the Sble enzyme is capable to convert (acetylated) bola amphiphilic glycolipids into (acetylated) acidic sophorolipids and (acetylated) acidic glucolipids while (acetylated) saccharides are released from the reaction.

Moreover, the present invention relates to the use of an Sble enzyme to convert (acetylated) bola sophorolipids into (acetylated) lactonic sophorolipids while releasing (acetylated) saccharides. It also relates to the use of an Sble enzyme to convert (acetylated) bola sophorolipids into (acetylated) acidic sophorolipids while releasing (acetylated) saccharides.

Moreover, the present invention relates to the use of an Sble enzyme to convert (acetylated) bola sophorolipids into (acetylated) lactonic sophorolipids while releasing (acetylated) sophorose and/or (acetylated) glucose. It also relates to the use of an Sble enzyme to convert (acetylated) bola sophorolipids into acidic sophorolipids and (acetylated) sophorose and/or glucose.

The present invention further relates to the surprising finding that yeasts strains which comprise a non-functional and/or dysfunctional Sble enzyme and/or which do not contain a (functional) sble gene, are capable to produce acetylated bola amphiphilic glycolipids.

It further relates to the surprising finding that yeasts strains which comprise a non-functional and/or dysfunctional At1 enzyme and/or in which the at1 gene is absent and/or disabled are capable to produce acetylated (bola) amphiphilic glycolipids, and, that surprisingly yeast strains which comprise additional non-functional and/or dysfunctional acetyltransferase enzymes At2 and At3 and/or in which the at2 and at3 genes are absent and/or disabled in addition to the At1 acetyltransferase enzyme/gene, produce non-acetylated (bola amphiphilic) glycolipids.

The present invention further relates to yeasts strains which in addition to comprise a non-functional and/or dysfunctional Sble enzyme additionally comprise a non-functional and/or dysfunctional UgtB1 enzyme and/or in which the ugtB1 gene is removed and/or disabled and which strains produce (acetylated) bola glucolipids.

The term ‘non- or dysfunctional’ means in general an enzyme or a fragment or a variant thereof, as described above, which is not functioning ‘normally’, and/or, has no (non-functional) or an impaired activity (dysfunctional). The term thus refers to an enzyme which is: a) not functional because it is not present, b) still present but non-functional or c) still present but with a weakened or reduced activity, whereby a weakened or reduced activity is an activity that is significantly less (p<0.05) than 90%, 80%, 70%, 60% or 50%, 40% or 30%, preferably less than 20%, more preferably less than 10%, even more preferably less than 5% such as less than 4%, 3%, 2% or 1% of the activity of the corresponding wild-type enzyme.

Situation a) wherein said enzyme or a fragment or a variant thereof is not functional because it is not present, situation b) is still present but non-functional or situation c) is still present but with a weakened or reduced activity, can be obtained through any known means to avoid, reduce and/or silence the transcription and/or translation of the nucleic acid sequence encoding said enzyme or through any known means to impair enzyme activity. For example, but not limited to, by knock out; by insertion of a nucleic acid fragment containing a marker gene or any other nucleotide fragment in the target gene resulting impaired transcription or translation of the nucleic acid sequence encoding said enzyme; through the usage of CRISPR; through homologous recombination; through siRNA; through CRISPRi; through the use of riboswitches; through recombineering; through ssDNA mutagenesis; through RNAi, miRNA, or asRNA; through mutating the enzyme or the nucleic acid sequence encoding said enzyme; through transposon mutagenesis; by disruption of (the function of) a necessary regulator/activator protein; through interference with the cellular synthesis of the target enzyme or of an activator/regulator; through the use of one or more aptamers; through the use of one or more ribozymes; through the use of antibodies, amino acids, peptides or any small molecules that interfere with transcription, translation, the synthesis of an active enzyme or enzyme activity; through the use of an oligoribonucleotide sequence such a dsRNA used to initiate RNA interference (RNAi) or an anti-sense nucleic acid; through the introduction of point mutations; through the usage of truncated, modified or mutated enzymes; through the usage of inhibitors or antibodies; through mutation (spontaneous, induced and/or directed, point mutation, deletion, frameshift, insertion or any other type of mutation); . . . or any other means known to a skilled person.

The term ‘disabled’ in the context of a gene means in general a gene or a fragment or a variant thereof, which is not functioning ‘normally’, and/or, has no or an impaired activity. The term thus refers to a gene which is: a) not functioning because it is not present, b) still present but not functioning or c) still present but with a weakened, reduced or altered activity. Situation a) wherein said gene or a fragment or a variant thereof is not functioning because it is not present, situation b) is still present but not functioning or situation c) is still present but with a weakened or reduced activity, can be obtained through any known means to avoid, reduce, alter and/or silence the transcription and/or translation of the nucleic acid sequence encoding said enzyme. For example, but not limited to, by knock out; by insertion of a nucleic acid fragment containing a marker gene or any other nucleotide fragment in the target gene resulting impaired transcription or translation of the nucleic acid sequence encoding said enzyme; by promoter engineering, by removal of the promotor, by switching promotors, by Kozak sequence engineering, by removal of the Kozak sequence, by switching Kozak sequences, through RBS (ribosomal binding site) engineering, by removal of the RBS, by switching RBS sequences, by UTR (untranslated region) engineering, by removal of the UTR, by switching UTR sequences, through the usage of CRISPR; through homologous recombination; through siRNA; through CRISPRi; through the use of riboswitches; through recombineering; through ssDNA mutagenesis; through RNAi, miRNA, or asRNA; through mutating the nucleic acid sequence encoding said enzyme; through transposon mutagenesis; by disruption of (the function of) a necessary regulator/activator protein; through interference with the cellular synthesis of the target enzyme or of an activator/regulator; through the use of one or more aptamers; through the use of one or more ribozymes; through the use of antibodies, amino acids, peptides or any small molecules that interfere with transcription, translation or the synthesis of an active enzyme; through the use of an oligoribonucleotide sequence such a dsRNA used to initiate RNA interference (RNAi) or an anti-sense nucleic acid; through the introduction of point mutations; through the usage of truncated, modified or mutated enzymes; through the usage of inhibitors or antibodies; through mutation (spontaneous, induced and/or directed, point mutation, deletion, frameshift, insertion or any other type of mutation); . . . or any other means known to a skilled person. The term ‘disabling’ means the act of rendering a disabled gene.

The term ‘removed’ in the context of a gene means in general a gene or a fragment or a variant thereof, which is, in whole or in part, removed from the genomic DNA. Such a removal can be obtained through any known means, for example, but not limited to, by knockout of the coding sequence through homologous recombination, by knockout of the gene through homologous recombination; by knockout of the coding sequence through the use of CRISPR technology, by knockout of the gene through the use of CRISPR technology; or any other means known to a skilled person. The term ‘removing’ means the act of rendering a removed gene.

The term ‘reduced expression’ is an expression that is significantly less (p<0.05) than 90%, 80%, 70%, 60% or 50%, 40% or 30%, preferably less than 20%, more preferably less than 10%, even more preferably less than 5% such as less than 4%, 3%, 2% or 1% of the expression of the corresponding wild-type gene. Such reduced expression can be obtained through any known means to avoid, reduce, alter and/or silence the transcription and/or translation of the nucleic acid sequence encoding said enzyme. For example, but not limited to, by knock out; by knock-down, by insertion of a nucleic acid fragment containing a marker gene or any other nucleotide fragment in the target gene resulting impaired transcription or translation of the nucleic acid sequence encoding said enzyme; by promoter engineering, by removal of the promotor, by switching promotors, by Kozak sequence engineering, by removal of the Kozak sequence, by switching Kozak sequences, through RBS (ribosomal binding site) engineering, by removal of the RBS, by switching RBS sequences, by UTR (untranslated region) engineering, by removal of the UTR, by switching UTR sequences, through the usage of CRISPR; through homologous recombination; through siRNA; through CRISPRi; through the use of riboswitches; through recombineering; through ssDNA mutagenesis; through RNAi, miRNA, or asRNA; through mutating the nucleic acid sequence encoding said enzyme; through transposon mutagenesis; by disruption of (the function of) a necessary regulator/activator protein; through interference with the cellular synthesis of the target enzyme or of an activator/regulator; through the use of one or more aptamers; through the use of one or more ribozymes; through the use of antibodies, amino acids, peptides or any small molecules that interfere with transcription, translation or the synthesis of an active enzyme; through the use of an oligoribonucleotide sequence such a dsRNA used to initiate RNA interference (RNAi) or an anti-sense nucleic acid; through the introduction of point mutations; through the usage of truncated, modified or mutated enzymes; through the usage of inhibitors or antibodies; through mutation (spontaneous, induced and/or directed, point mutation, deletion, frameshift, insertion or any other type of mutation); . . . or any other means known to a skilled person.

The term ‘variant’ refers to a protein or peptide or polypeptide as depicted by SEQ ID N° 2, SEQ N° 4, SEQ ID N° 6, SEQ N° 8 and/or SEQ N° 62 having at least 34% sequence identity, preferably having at least 51-70% sequence identity, more preferably having at least 71-90% sequence identity or most preferably having at least 91, 92, 93, 94, 95, 96, 97, 98 or 99% sequence identity with SEQ ID N° 2 or with a fragment thereof, and that retains said enzymatic activity.

The percentage of amino acid sequence identity is determined by alignment of the two sequences and identification of the number of positions with identical amino acids divided by the number of amino acids in the shorter of the sequences×100.

The latter ‘variants’ may also differ from the proteins as depicted by SEQ ID N° 2, SEQ N° 4, SEQ ID N° 6, SEQ N° 8 and/or SEQ N° 62 only in conservative substitutions and/or modifications, such that the ability of the activity is retained. A “conservative substitution” is one in which an amino acid is substituted for another amino acid that has similar properties, such that one skilled in the art of protein chemistry would expect the nature of the protein to be substantially unchanged. In general, the following groups of amino acids represent conservative changes: (1) Ala, Pro, Gly, Glu, Asp, Gln, Asn, Ser, Thr; (2) Cys, Ser, Tyr, Thr; (3) Val, Ile, Leu, Met, Ala, Phe; (4) Lys, Arg, His; and (5) Phe, Tyr, Trp, His.

Variants may also (or alternatively) be proteins as described herein modified by, for example, the deletion or addition of amino acids that have minimal influence on the enzymatic activities as defined below, secondary structure and hydropathic nature of the enzyme.

Furthermore, the term variants also refers to any glycosylated protein or any protein modified in any other way as depicted by SEQ ID N° 2, SEQ N° 4, SEQ ID N° 6, SEQ N° 8 and/or SEQ N° 62 or fragments thereof. A non-limitative list of such protein modifications: acetylation, acylation, ADP-ribosylation, amidation, covalent attachment, cross-linking, cyclization, disulfide bond formation, demethylation, formation of covalent cross-links, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, lipid attachment, sulfation, selenoylation, transfer-RNA mediated addition of amino acids to proteins, such as arginylation, and ubiquitination, . . . .

Hence, orthologues and paralogues, or any gene in other genera and species (than the strain Starmerella bombicola ATCC 22214 from which SEQ ID N° 1-8 and 61-62 are derived) which encode for polypeptides having the described activities, are part of the present invention.

The term ‘an Sble enzyme’ relates to the enzyme which is previously denominated as a ‘lactonase’ or ‘Starmerella bombicola lactone esterase’ and is described in detail in WO2013/092421. The Sble enzyme of the present invention thus relates to a polypeptide comprising an amino acid sequence given by SEQ ID N° 2, or a fragment thereof retaining the above-described enzymatic activity (i.e. ‘the transesterification and/or hydrolysis activity on (acetylated) bola amphiphilic glycolipid compounds, more specifically the conversion of (acetylated) bola sophorolipids/(acetylated) bola glucolipids into (acetylated) lactonic sophorolipids/(acetylated) lactonic glucolipids respectively while releasing (acetylated) saccharides such as (acetylated) sophorose and/or (acetylated) glucose and/or the conversion of (acetylated) bola sophorolipids/(acetylated) bola glucolipids into (acetylated) acidic sophorolipids/(acetylated) acidic glucolipids while releasing (acetylated) saccharides such as (acetylated) sophorose and/or (acetylated) glucose, or a variant thereof having at least 34% sequence identity with SEQ ID N° 2 and having said enzymatic activity.

The nucleic acid sequence as depicted by SEQ ID N° 1 corresponds to the open reading frame of 1233 base pairs which encodes for the polypeptide sequence of the Sble enzyme of the present invention as depicted by the 410 amino acid sequence SEQ ID N° 2:

SEQ ID No 1:
ATGCTGGCTCTGTTTTTTTCGCTTGCGCCTCTACTTTCTCAAGCTCTCCCTTTAGGCTATACTGCGGCCCCCGCTG
AATCATTCTATTTTTGGCCAGAGAACATATCCAGCCTCCAAGCTGGCGAGATTTTTAGAAAACGGGAACTCTTA
ACTCTCCCAGACATCTTTGACTTTGGCCCTAATCTGGAAAAGGTCGTACAAGTGGCTTACAAAACCCGTCTCAC
CGATGGCAATGACTCGTTTTCCATCGCCAGTATCTTTATCCCTAAGAATCCAAGCCCAGAACTCAAACTTTACTC
TTATCAGACGTTTGAGGATGCCGTGCAGCTTGATTGTGCCCCAAGCTATGCTTTAGAAGTGGGTAACAAGTCC
AGCAACTATCTTCCTGTCACTAGCAATTTATCTGCCATCAGTCGAGAACTTGAGAAAGGACGTCACTGCATTAT
CCCTGATCACGAGGGCTATATTTCAGGATTCTTTGCAGGACGGCAGGAGGGATATGCTGGTTTAGACGGAATT
CGCGCTGCTCGAAACTATCTCAATGGCACCAACGAGACCCCAATTGGTATCTTCGGATACAGTGGAGGTGCAC
AAGCAACGGCCTGGATTGTTGATTTGCATGACGAGTATGCTCCTGACTTGAACTTTGTTGGAACAGTTTCTGGA
GGCACTTTGGTTGACGCTTGGGGCACTTTTCAGTATATCGACTATCCGAAGGTGTATCTAAAGGGCAGCATTCT
TATCATGTATACGGGTCTTTTTTCAGGTTATCCAGCTCAATTTGAGGTGATTTGGCCATATATTGAGCCTGTAAT
TCAAGAAAACATGCTACTGCTACGTTTGGCGCCGAATGATTGTAACCAAAGCCCGATACTTCAAGGTTACAACA
ATTCAATCATGGCCGGTATACATGTGGACCTTCCCGAATTCCCTGCTTCTAAGTACATATTCCAGCACGAGTCCC
TCCTTGCCAACTACAGCGTAGTGCCAGTTTCCACACCGAAGTTTCCTCGCTACATGTACCATGGTGGATCTGAT
GAGTTGGCCAAATTGAGCCTTGTCGAGCAGTATGTTGATCAACAATGGAATACCGGCGCTAATCTCACCTTCGT
GGTGTATCCGGGTCTTCTTCATGACGAGACGGCTTACCGTGGCTTTGATGCCGCGATGGATTGGCTTGATGCC
CAGCTCGATAGTGGATACCTTCCACCTGTAAACTCAACTCATACATGA
SEQ ID No 2:
MLALFFSLAPLLSQALPLGYTAAPAESFYFWPENISSLQAGEIFRKRELLTLPDIFDFGPNLEKVVQVAYKTRLTDGND
SFSIASIFIPKNPSPELKLYSYQTFEDAVQLDCAPSYALEVGNKSSNYLPVTSNLSAISRELEKGRHCIIPDHEGYISGFFA
GRQEGYAGLDGIRAARNYLNGTNETPIGIFGYSGGAQATAWIVDLHDEYAPDLNFVGTVSGGTLVDAWGTFQYID
YPKVYLKGSILIMYTGLFSGYPAQFEVIWPYIEPVIQENMLLLRLAPNDCNQSPILQGYNNSIMAGIHVDLPEFPASKY
IFQHESLLANYSVVPVSTPKFPRYMYHGGSDELAKLSLVEQYVDQQWNTGANLTFVVYPGLLHDETAYRGFDAAM
DWLDAQLDSGYLPPVNSTHT

The term ‘fragment’ further refers to a protein or peptide or polypeptide containing fewer amino acids than the amino acid sequence as depicted by SEQ ID N° 2 and that retains said enzymatic activity i.e. ‘the transesterification and/or hydrolysis reaction on bola amphiphilic glycolipid compounds’. Such fragment can—for example—be a protein with a deletion of 10% or less of the total number of amino acids at the C- and/or N-terminus.

Moreover, the present invention relates to the usage of an Sble enzyme to convert tetra-acetylated bola sophorolipids into di-acetylated lactonic sophorolipids while releasing (acetylated) saccharides such as (acetylated) glucose and/or (acetylated) sophorose.

Moreover, the present invention relates to the usage of an Sble enzyme to convert non-, mono-, di- and/or tri-acetylated bola sophorolipids into non-, mono and/or di-acetylated lactonic sophorolipids while releasing non-, mono- or di-acetylated sophorose and/or glucose.

Moreover, the present invention relates to the usage of an Sble enzyme to convert tetra-acetylated bola sophorolipids into di-acetylated acidic sophorolipids while releasing (acetylated) saccharides such as (acetylated) glucose and/or (acetylated) sophorose.

Moreover, the present invention relates to the usage of an Sble enzyme to convert non-, mono-, di- and/or tri-acetylated bola sophorolipids into non-, mono- and/or di-acetylated acidic sophorolipids while releasing (acetylated) saccharides such as non- and/or mono-acetylated glucose and/or non-, mono- and/or di-acetylated sophorose.

Furthermore, the present invention relates to the usage of a modified yeast strain, which comprises a non- and/or dysfunctional Sble enzyme and/or in which the sble gene is removed and/or disabled and which strain is able to produce (acetylated) bola amphiphilic glycolipids such as bola sophorolipids and/or bola glucolipids.

The term ‘a modified yeast strain’ relates to a yeast strain modified in any way so that the Sble enzyme is non- or dysfunctional and/or where the sble gene is removed and/or disabled.

More specifically, the present invention relates to the use of a modified yeast strain as described above wherein said bola sophorolipids have an acetylation degree of 0, 1, 2, 3 or 4.

With the term ‘an acetylation degree’ of 4 is meant that all of the four glucose moieties present in the bola SLs are acetylated.

Furthermore, the present invention relates to the usage of a modified yeast strain, which comprises a non- or dysfunctional At1 enzyme and/or which does not contain a (functional) at1 gene, encoded in the SL biosynthetic gene cluster and which strains are surprisingly able to produce acetylated (bola) amphiphilic glycolipids more specifically acetylated (bola) sophorolipids and/or acetylated (bola) glucolipids and wherein said acetylated bola sophorolipids and/or glucolipids have an acetylation degree of 0, 1 or 2. With the term ‘an acetylation degree of 0, 1 or 2 is meant that zero, one or two glucose moieties respectively present in the bola amphiphilic glycolipids are acetylated.

The term ‘a modified yeast strain’ relates to a yeast strain modified in any way—as is already described above for the Sble enzyme—so that the At1 enzyme is non- or dysfunctional as described above and/or the at1 gene is removed and/or disabled.

The term ‘an Acetyltransferase (At1) enzyme 1’ relates to the enzyme previously described in detail in in detail in WO2012/080116 and by Saerens et al. (2011b) and Saerens et al. (2015). This At1 enzyme is referred to as ‘At1’ in the present invention and is thus responsible for acetylation of glycolipids produced by S. bombicola (Saerens et al. (2015)). The At1 enzyme of the present invention thus relates to a nucleic acid sequence as depicted by SEQ ID N° 3 corresponding to an open reading frame of 780 base pairs which encodes for the polypeptide comprising an amino acid sequence given by SEQ ID N° 4, or a fragment thereof retaining the above-described enzymatic activity (i.e. ‘the acetylation of (bola) amphiphilic glycolipid compounds, more specifically the acetylation of (bola) sophorolipids and/or (bola) glucolipids) and thus relates to a nucleic acid sequence as depicted by SEQ ID N° 3 corresponding to an open reading frame of 780 base pairs which encodes for the polypeptide comprising an amino acid sequence given by SEQ ID N° 4, or a fragment thereof retaining the enzymatic activity or a variant thereof having at least 34% sequence identity with SEQ ID N° 4 and having said enzymatic activity.

SEQ ID No 3:
atggttgtaaactcctcgaaggaccctcaaaacaaaggaatgactcctagaaaagaaattgaccaggaaatggtctcttgggccaaaaaaaac
ctcaaaaacacccctggcaatgaaaactatgagaagatggtctcaggagttccttacaatccatacgatccagatcttatgtttagagccctgg
ctactagtgagaaagttagggagttcaataccattgcaagtgaaagtcgtacttttgagtcaaatcacgctgcttatatcaagaaggtcgagat
tctcaaagacacttttggtcaaacaaaggatattgtctggctgaccgctccattctcagttgattttggattcaacatcagcgtaggcgagcac
ttttacgccaacttcaacgtttgcttcttggactcggctccaataatctttggtgatgaggtgattgtagggcccaatacaacgttcgtgactg
cgactcatcctattagccccgagaaacgtgcgaggagaattgtgtatgctcttcctatcaaggtggggaataatgtatggattggtgcgaatgt
gactgtcctgccgggtgttacgattggagatggctcaacaattgcggctggtgctgtcgttcgagaagatgttcctcctcgtactgtggtggga
ggagtccctgcgcgaatcctcaagcatattccagaggaggatcccgacgaggctgaaggagaggaactggaattccttcttccagttgaaatga
acgtcaataccgctaaccagaaggtctag
SEQ ID No 4:
MVVNSSKDPQNKGMTPRKEIDQEMVSWAKKNLKNTPGNENYEKMVSGVPYNPYDPDLMFRALATSEKVREFNT
IASESRTFSNHAAYIKKVEILKDTFGQTKDIVWLTAPFSVDFGFNISVGEHFYANFNVCFLDSAPIIFGDEVIVGPNTTF
VTATHPISPEKRARRIYALPIKVGNNVWIGANVTVLPGVTIGDGSTIAAGAVVREDVPPRTVVGGVPARILKHIPEED
PDEAEGEELEFLLPVEMNVNTANQKV

The term ‘fragment’ further refers to a protein or peptide or polypeptide containing fewer amino acids than the amino acid sequence as depicted by SEQ ID N° 4 and that retains said enzymatic activity i.e. ‘the acetylation of (bola) amphiphilic glycolipid compounds, more specifically the acetylation of (bola) sophorolipids and/or (bola) glucolipids’. Such fragment can—for example—be a protein with a deletion of 10% or less of the total number of amino acids at the C- and/or N-terminus.

It further relates to the use of a modified yeast strain as described above which further comprises two additional non- or dysfunctional glycoside O-acetyltransferase enzymes (At2 and At3) and/or a combination of both enzymes being rendered non- or dysfunctional besides the dysfunctional At1 enzyme as described above, to produce fully non-acetylated (bola) amphiphilic glycolipids such as (bola) sophorolipids/glucolipids. The term ‘glycoside O-acetyltransferase enzymes’ relates to enzymes referred to as At2 and At3 in the present invention which are responsible for acetylation of (bola) amphiphilic glycolipids.

The term ‘a modified yeast strain’ relates to a yeast strain modified in any way so that the At2 and/or At3 enzyme are non- or dysfunctional as described above and/or in which the at2 and/or at3 genes have been removed and/or disabled.

It further relates to the use of a modified yeast strain which comprises one of three non- or dysfunctional acetyltransferase enzymes (At1, At2, At3) and/or a combination of the three enzymes being rendered non- or dysfunctional, to produce non-acetylated glycolipids such as non-acetylated lactonic SLs, non-acetylated acidic glucolipids, non-acetylated acidic sophorolipids, non-acetylated bola sophorolipids, non-acetylated bola sophorolipids, non-acetylated bola glucolipids etc. The term ‘a modified yeast strain’ relates to a yeast strain modified in any way—as is described above for the Sble enzyme—so that the At1, At2 and/or At3 enzyme is/are non- or dysfunctional.

The At2 enzyme of the present invention thus relates to a nucleic acid sequence as depicted by SEQ ID N° 5 corresponding to an open reading frame of 663 base pairs which encodes for the polypeptide comprising 220 amino acids with a sequence given by SEQ ID N° 6, or a fragment thereof retaining the above-described enzymatic activity (i.e. ‘the acetylation of (bola) amphiphilic glycolipid compounds, more specifically the acetylation of (bola) sophorolipids and/or (bola) glucolipids’ or a fragment thereof retaining the enzymatic activity or a variant thereof having at least 34% sequence identity with SEQ ID N° 6 and having said enzymatic activity.

SEQ ID No 5:
ATGCCTAGCGGAGCCCCAAGAATCGAGTACAATTGGGACCTGATCAAGTGGGCTCGCGAAAATTTGTCCCATT
TGCCGGTAGATGATGACAACTATCACCGGATGATTAGTGGGTTGCCATATGAGGCAACCCGCACAGATTATTC
GCGCCATCGAATAGAGTCCCATGAATTACTTCTAGAATACTTGAATATGAAACTGAAGGACTTCGCTACGTTGG
AAAAATATAATCAGGCGCGAGCAGATTTGCTTTCAAAGGTGTTTGGCTCCATGGGCACAAACTGCTTCATTGA
GCAACACCTATTTGTAGATTATGGTTGCAACATTAAAGTCGGCAATAACTITTATGCGAACAACAACCTTACAA
TGCTCGATTGCTCTGTCATTGAGATTGGCGACAATGTGTTTTTTGGACCTAATGTAACAATCACTACGGCATCTC
ACCCGTTGGAATCGAAGCCTAGGGCCGAAGGGGTCGAATTCGCTTTCAATGTCAAAATCGGAAACAACGTCTG
GATAGGTTCCAACGCTGTGGTCTTGCCGGGAGTTACCATTGGAGATGACGTAGTCGTTGCAGCTGGCGCAGTG
GTCAACAAGGATGTGCCCCCTTCAGTCGTAGTGGGCGGTGTCCCGGCGAAAATTCTTAAGCAAATCCAGAATT
GA
SEQ ID No 6:
MPSGAPRIEYNWDLIKWARENLSHLPVDDDNYHRMISGLPYEATRTDYSRHRIESHELLLEYLNMKLKDFATLEKYN
QARADLLSKVFGSMGTNCFIEQHLFVDYGCNIKVGNNFYANNNLTMLDCSVIEIGDNVFFGPNVTITTASHPLESKP
RAEGVEFAFNVKIGNNVWIGSNAVVLPGVTIGDDVVVAAGAVVNKDVPPSVVVGGVPAKILKQIQN

The term ‘fragment’ further refers to a protein or peptide or polypeptide containing fewer amino acids than the amino acid sequence as depicted by SEQ ID N° 6 and that retains said enzymatic activity i.e. ‘the acetylation of (bola) amphiphilic glycolipid compounds, more specifically the acetylation of (bola) sophorolipids and/or (bola) glucolipids’. Such fragment can—for example—be a protein with a deletion of 10% or less of the total number of amino acids at the C- and/or N-terminus.

The At3 enzyme of the present invention thus relates to a nucleic acid sequence as depicted by SEQ ID N° 7 corresponding to an open reading frame of 747 base pairs which encodes for the polypeptide comprising an amino acid sequence given by SEQ ID N° 8 of 248 amino acids, or a fragment thereof retaining the above-described enzymatic activity (i.e. ‘the acetylation of (bola) amphiphilic glycolipid compounds, more specifically the acetylation of (bola) sophorolipids and/or (bola) glucolipids’ or a variant thereof having at least 34% sequence identity with SEQ ID N° 8 and having said enzymatic activity.

SEQ ID No 7:
ATGTTGCCTGCAACAGAAATCGATAGAGAACTCGTGCAATGGGCTCGCGAAAATCTTCCAAACCTCCCTCAAA
GCACACATTATGACAAGCAGATTAGTGGCATGCTGATCAAGCCCAAATGGTCCTCAATGGTTCACGAGACAAA
GATGAAACAGCTCACAAGGGACTATGACAGCATCAATCTCAACCATTTCAGCTCTGTGGCGAAATACTTTGAG
GCCAGGACAAGCTTTATCCAGAAGCATCTCCTCGGCAAAACAGGAAAGAGAGTCTACCTCGAATCCCCAGTTC
ACATCAATCACGGATACAATATATCGGTAGGCGAAAACTTCTATTGCAACTTTAATTGCATATTTCTCGACTGGT
CCATAATCAGAATTGGCGACAACGTTGCGATTGGCCCCAACTGTACCTTAAGTTGCATTAATCATCCCTTGAGT
GGCGATGATCGCAAAAATGGTGCGGGTTTATACGCTTTCCCTATCTTTATCGATGACAATGTCTGGATAGGGG
CGAACTGTGTGATTCTTTCAGGGATTCATGTTGCTGAAGGGTCGGTTGTCGCCGCAGGATCGGTAGTGACAAA
AAGTGTGCCCCCTCATGTTATCGTGGCTGGCAATCCTGCGAAGATCATTGCGAAGGCAACAGACCGACGACTT
CGGGCTGCCGCAGAGGATTCATCCTCCCCAGAATCTTCGGACGCCGAAGAGAGCTACATGTTCATTACCAAGA
CTGCGGATCCCTGA
SEQ ID No 8:
MLPATEIDRELVQWARENLPNLPQSTHYDKQISGMLIKPKWSSMVHETKMKQLTRDYDSINLNHFSSVAKYFEAR
TSFIQKHLLGKTGKRVYLESPVHINHGYNISVGENFYCNFNCIFLDWSIIRIGDNVAIGPNCTLSCINHPLSGDDRKNG
AGLYAFPIFIDDNVWIGANCVILSGIHVAEGSVVAAGSVVTKSVPPHVIVAGNPAKIIAKATDRRLRAAAEDSSSPESS
DAEESYMFITKTADP

The term ‘fragment’ further refers to a protein or peptide or polypeptide containing fewer amino acids than the amino acid sequence as depicted by SEQ ID N° 8 and that retains said enzymatic activity i.e. ‘the acetylation of (bola) amphiphilic glycolipid compounds, more specifically the acetylation of (bola) sophorolipids and/or (bola) glucolipids’. Such fragment can—for example—be a protein with a deletion of 10% or less of the total number of amino acids at the C- and/or N-terminus.

Hence, the present invention also relates to an isolated acetyltransferase having an amino acid sequence given by SEQ ID N° 6 or SEQ ID N° 8 and denominated as acetyltransferase 2 (At2) and acetyltransferase 3 (At3), respectively.

Furthermore, the present invention relates to the use of a modified yeast strain as described above which further comprises a non- or dysfunctional glucosyltransferase UgtB1 enzyme or in which the ugtB1 gene has been removed and/or disabled and wherein acetylated and/or non-acetylated bola glucolipids are produced instead of bola sophorolipids. The term ‘UgtB1 enzyme’ relates to the enzyme described in detail by Saerens et al. (2011c and 1015) with a glycosylation activity of (bola) glucolipids towards (bola) sophorolipids. The UgtB1 enzyme of the present invention thus relates to a nucleic acid sequence as depicted by SEQ ID N° 61 corresponding to the open reading frame of 1299 base pairs which encodes for the polypeptide sequence of the UgtB1 enzyme as depicted by the 432 amino acid sequence SEQ ID N° 62 or a fragment thereof retaining the enzymatic activity or a variant thereof having at least 34% sequence identity with SEQ ID N° 62 and having said enzymatic activity. The term ‘a modified yeast strain’ relates to a yeast strain modified in any way so that the UgtB1 enzyme encoded in the SL biosynthetic gene cluster (Saerens et al. (2011c)) is non- or dysfunctional as described above.

SEQ ID No 61:
ATGGCCATCGAGAAACCAGTGATAGTTGCTTGTGCCTGCCCACTAGCGGGGCACGTGGGCCCAGTGCTCAGCC
TGGTCCGCGGTCTACTCAATAGAGGATATGAGGTGACTTTCGTAACAGGGAACGCATTCAAGGAGAAAGTTAT
TGAGGCAGGATGCACTTTCGTCCCTCTCCAAGGACGAGCTGACTACCATGAATACAATCTCCCTGAAATCGCTC
CAGGATTGCTCACGATTCCTCCAGGCCTTGAGCAGACCGGTTACTCAATGAATGAGATTTTTGTGAAGGCGATT
CCTGAGCAGTACGATGCACTTCAAACTGCTCTAAAACAGGTTGAGGCTGAAAATAAATCAGCTGTGGTGATTG
GCGAGACCATGTTTCTAGGGGTGCATCCGATATCACTGGGTGCCCCAGGTCTCAAGCCCCAAGGCGTAATCAC
GTTAGGAACTATTCCGTGCATGCTGAAAGCAGAGAAGGCGCCTGGAGTTCCTAGTCTTGAGCCAATGATTGAT
ACTTTAGTGCGGCAACAAGTATTTCAACCAGGAACTGACTCTGAGAAGGAGATCATGAAGACGCTCGGGGCC
ACGAAGGAGCCCGAATTTCTCCTGGAGAATATATACAGCAGCCCTGACAGATTTTTGCAACTGTGCCCTCCATC
TCTTGAATTTCACTTGACTTCGCCTCCTCCTGGCTTCTCGTTCGCTGGTAGTGCACCGCATGTAAAGTCTGCTGG
ATTAGCAACTCCACCTCACCTGCCGTCTTGGTGGCCTGATGTGCTGAGTGCGAAGCGTCTGATTGTTGTTACAC
AAGGAACAGCAGCCATCAACTATGAAGATCTGCTCATTCCAGCATTGCAGGCCTTTGCTGACGAAGAAGACAC
TCTCGTAGTTGGTATATTGGGCGTCAAAGGGGCGTCACTTCCTGATAGCGTTAAAGTTCCTGCAAACGCTCGAA
TTGTTGATTATTTTCCTTACGATGAGCTACTACCGCATGCCTCTGTTTTCATATACAACGGTGGATACGGAGGTC
TGCAGCACAGTTTGAGCCATGGCGTTCCCGTCATCATCGGAGGAGGAATGTTGGTAGACAAGCCAGCTGTTGC
TTCACGAGCTGTATGGGCTGGTGTTGGTTATGATCTTCAAACCTTGCAGGCAACTTCTGAGCTAGTCTCCACGG
CCGTTAAGGAGGTGTTGGCTACTCCCTCGTATCACGAGAAAGCCATGGCAGTCAAGAAAGAGCTTGAAAAATA
CAAGTCTCTTGATATTCTAGAGTCGGCAATTAGTGAATTAGCTTCTTAA
SEQ ID No 62
MAIEKPVIVACACPLAGHVGPVLSLVRGLLNRGYEVTFVTGNAFKEKVIEAGCTFVPLQGRADYHEYNLPEIAPGLLTI
PPGLEQTGYSMNEIFVKAIPEQYDALQTALKQVEAENKSAVVIGETMFLGVHPISLGAPGLKPQGVITLGTIPCMLKA
EKAPGVPSLEPMIDTLVRQQVFQPGTDSEKEIMKTLGATKEPEFLLENIYSSPDRFLQLCPPSLEFHLTSPPPGFSFAG
SAPHVKSAGLATPPHLPSWWPDVLSAKRLIVVTQGTAAINYEDLLIPALQAFADEEDTLVVGILGVKGASLPDSVKVP
ANARIVDYFPYDELLPHASVFIYNGGYGGLQHSLSHGVPVIIGGGMLVDKPAVASRAVWAGVGYDLQTLQATSELV
STAVKEVLATPSYHEKAMAVKKELEKYKSLDILESAISELAS

The term ‘fragment’ further refers to a protein or peptide or polypeptide containing fewer amino acids than the amino acid sequence as depicted by SEQ ID N° 62 and that retains said enzymatic activity i.e. ‘the glycosylation of (bola) amphiphilic glucolipid compounds, more specifically the glucosylation of (bola) glucolipids. Such fragment can—for example—be a protein with a deletion of 10% or less of the total number of amino acids at the C- and/or N-terminus.

More specifically, and already mentioned above, the present invention further relates to the usage of a modified yeast strain as described above wherein said yeast strain is a yeast strain selected from the group consisting of Starmerella bombicola (previously Candida) (Spencer et al., 1970), Starmerella apicola (Gorin et al., 1961) (previously Candida), which was initially identified as T. magnolia, Wickerhamiella domericqiae (Chen et al., 2006), Pseudohyphozyma bogoriensis sp. (previously Rhodotorula or Candida) (Tulloch et al., 1968), Starmerella batistae (Konishi et al., 2008) (previously Candida), Starmerella (previously Candida) floricola (Imura et al., 2010), Starmerella (previously Candida) batistae, Candida riodocensis, Candida tropicalis, Starmerella stellata (previously Candida) and Candida sp. NRRL Y-27208 (Kurtzman et al., 2010), Starmerella kuoi (Kurtzman, 2012) (previously Candida), Candida gropengiesseri, Candida magnoliae, Candida antarctica, Pseudozyma antarctica, Candida tropicalis, Candida lipolytica and any other SL producing strain (of the Starmerella clade).

Moreover, the present invention relates to the usage of a modified yeast strain as described above wherein the activities of the Sble enzyme, the UgtB1 enzyme and/or the acetyltransferase enzymes At1, AT2 and At3 and/or their encoding genes are disabled.

### EXAMPLES

### Example 1

### Production of Acetylated Bola Glycolipids.

### Material and Methods

### Strains and Cultivation Methods

Cloning experiments and plasmid maintenance was performed with Escherichia coli top 10 cells. E. coli cells were grown in Luria-Broth medium (37° C., 10 g/l trypton, 5 g/l yeast extract, 5 g/l sodium chloride and if required 15 g/l agar; Sigma-Aldrich) supplemented with 100 mg/L ampicillin (LB-amp; MP Biomedicals) when applicable. Wild type S. bombicola (WT; ATCC 22214) and a URA3 auxotrophic mutant strain (PT36) were used during this study (Lodens et al., 2018). Two existing S. bombicola strains developed in the past were also included: the single deletion strain Δsble (Ciesielska et al. 2014) and the double deletion strain Δat1 Δsble (Van Bogaert et al., 2016 and WO2015/028278). Solid synthetic dextrose with complete supplement mixture without uracil (6.7 g/L Yeast nitrogen base without amino acids (Sigma-Aldrich), 20 g/L glucose (Cargill), 20 g/L agar Noble (Difco), 0.77 g/L complete supplement mixture without uracil (MP biomedicals)) and yeast extract peptone dextrose supplemented with hygromycin (20 g/L glucose (cargill), yeast extract (DSM), 20 g/L bactopepton (BD biosciences), agar (Biokar Diagnostics), 1 g/L Hygromycine B (Sigma-Aldrich) were used for selection for positive deletion mutants after transformation with a URA3 auxotrophic or a Hygromycine resistance marker, respectively.

For the glycolipid production experiments, the production medium as described by (Lang et al., 2000) was used. Precultures (5 ml) were inoculated from cryovials (1%) and incubated for 48 h (30° C., 200 rpm). Subsequently, shake flasks (n=3) containing 100 mL production medium were inoculated (1%) from precultures. Shake flasks were incubated for 240 h (30° C., 200 rpm). 37.5 g/L oleic acid (Sigma-Aldrich) was supplemented after 48 h of cultivation.

### Analytical Techniques

Cell dry weight (CDW) was measured by performing a centrifuge step to 1 ml of shake flask broth (5 min, 14 000 rpm) after which the supernatant was discarded. The biomass pellet was resuspended in a 0.9% (w/v) NaCl solution and centrifuged once again for 5 min at 14000 rpm. After this washing step, the supernatant was discarded and the biomass was placed in an oven at 60° C. for at least 50 hours in order to remove residual moisture. Finally, the net dry biomass was measured by gravimetrically methods again and the total CDW was determined and expressed in g dry biomass/L.

pH of SF broth samples was measured with a Five easy F20 Mettler Toledo pH/mV meter with two-point calibration.

Production samples were analysed by UPLC-HRMS (Thermo Scientific™ Exactive™ Plus Orbitrap Mass Spectrometer). Products were separated by UPLC according to (Van Renterghem et al., 2018). Sample preparation was performed on SF broth samples. Firstly, 70% EtOH (3:1, v/v) was added to the sample and vigorously vortexed for 5 min. Subsequently, a centrifugation step was performed (5 min, 14 000 rpm) on which the supernatants was filtered through a PES filter (0.2 μm, sartorius). Four in-house SL standards were analysed together with the production samples.

### Molecular Methods

Circular polymerase extension cloning (CPEC) pieces and the linear deletion cassettes were amplified with Primestar® GXL according to the manufacturer's instructions. Colony PCR was performed on E. coli and S. bombicola according to (De Graeve et al., 2019). S. bombicola colony PCRs were performed to analyse the 5′, 3′ and full overlap of the integration of the deletion cassette in the genome. CPEC was performed with Q5® Hifi DNA polymerase according to the manufacturer's instructions and as described in (Quan and Tian, 2009). CPEC assembly products and linear deletion cassettes were transformed via electroporation according to (De Graeve et al., 2019) into E. coli and S. bombicola, respectively. Sequencing of CPEC assembled plasmids was performed by Macrogen inc.

Three different deletion cassettes were constructed for subsequent gene deletion in S. bombicola (FIG. 3-5).

Genetic elements originate from the S. bombicola genome except the hygromycin B selection marker (HygroR) and the terminator of the Herpes simplex virus tyrosine kinase (tTK) terminator that were used as described by (Van Bogaert et al., 2008). In order to construct the deletion cassettes, fragments were first amplified and assembled with the aid of circular polymerase extension cloning (CPEC) plasmid assembly on a pJET vector backbone (pJET; Thermo scientific) (Quan and Tian, 2009). These plasmids were transformed into E. coli top 10 cells and positive colonies were selected from LB-amp and verified by colony PCR and subsequent DNA sequencing. Primers used for amplification of fragments, amplification origin of fragments and primers used for E. coli colony PCR are listed in Table 1. The disabling of the ugtB1 gene was achieved as described by Lodens et al. (2020)

### Results

Evaluation of Existing S. bombicola Strains

Recently performed biosurfactant production experiments as described under materials and methods with three S. bombicola strains developed and described in the past: Δsble (Ciesielska et al. 2014 and WO2013/092421), Δat1 Δsble (Van Bogaert et al., 2016 and WO2015/028278) and Δat1 Saerens et al. (2011b) resulted in two unexpected observations in contradiction with the art. The first observation relates to the surprising detection of masses corresponding to (acetylated) bola sophorolipids up to an acetylation degree of 4 in samples from the experiment with the Δsble strain described by (Ciesielska et al. 2014, Roelants et al. 2016 and WO2013/092421) and thus in contrast to these previous observations and reports where only acidic SLs were described to be produced. The second observation similarly relates to the surprising detection of acetylated (bola) sophorolipids up to an acetylation degree of 2 (mainly acetylation degree of 1) in the samples from the experiments with the Δat1 Δsble strain (Van Bogaert et al., 2016 and WO2015/028278) and the Δat1 strain Saerens et al. (2011b) and thus in contrast to these previous observations and reports. Both observations are in contradiction with the art and unexpected as the Δsble strain had been described to exclusively produce (acetylated) acidic SLs. Di-acetylated acidic SLs have been described to be the substrate of the Sble enzyme converting these into di-acetylated lactonic SLs (Ciesielska et al., 2014 and 2016). The Δat1 Δsble strain and Δat1 had been described not to produce any acetylated (bola) SLs, due to mutation of the at1 gene, which had been described as the (only) enzyme responsible for acetylation of sophorolipids. The production of acetylated bola sophorolipids had thus never been described. These findings were thus highly unexpected and additional experiments were performed.

As the existing strains were developed using restriction enzyme mediated methods, parts of the ORFs of the sble and at1 genes are still present in the modified strains described above, potentially resulting in residual enzyme activity. Novel S. bombicola strains were thus generated as described below with full deletion of the ORFs/coding sequences as described under materials and methods and their glycolipid production profile was evaluated after performing production experiments.

Creation and Evaluation of Novel S. bombicola Strains

The deletion cassettes described under materials and methods and shown FIGS. 3-5 were amplified from their respective plasmids and used for transformation into S. bombicola strains. Cassette 1 and 2 were used for the creation of S. bombicola strains containing a single gene deletion by homologous recombination, i.e. Δsble_full and Δat1_full, respectively. After successful verification of the gene deletions, cassette 3 was used for the deletion of at1 in the novel Δsble_full strain. Table 2 lists the created strains, the respective deletion cassette, the primers used for amplification of deletion cassettes, the primers used for colony PCR evaluation, the original strain and the obtained genotype.

The newly developed strains were evaluated for their production characteristics in shake flask (SF) experiments together with the S. bombicola wild type (WT) strain. A similar pH decline was observed in all SFs, starting at a pH of approximately 5.8, decreasing rapidly to a pH of about 3 around 48 h after inoculation, at which value it remained. A similar growth was observed for all strains with a maximum cell dry weight (CDW) of 19 g/L at 84 h after inoculation. Afterwards, the CDW remained constant for all strains except for the WT strain. This is mainly due to the fact that solid lactonic sophorolipids (SLs) remain with the cell pellet for the wild type causing a biased CDW determination. These lactonic SLs were clearly visible as a separate layer in the centrifuged SF broth samples gathered from the WT strain from 84 h till 240 h of production while no such layer was detected in the analogous SF samples from the Δat1, Δsble and Δat1 Δsble S. bombicola strains.

Production samples obtained at 180 h after inoculation were subjected to UHPLC-HRMS analysis. The results are described in the text below and summarized in Table 4.

The wild type S. bombicola produces predominantly C18:1 di-acetylated (diAc) lactonic SL (L SL) as expected.

The novel Δsble_full strain was evaluated, and the SL spectrum consists primarily of m/z values matching the monoisotopic masses of nAc C18:1 bola SL, mAc C18:1 bola SL, diAc C18:2 bola SL, diAc C18:1 bola SL, tri-acetylated (triAc) C18:1 bola SL, tetra-acetylated (tetraAc) C18:1 bola SL, tetraAc C18:0 bola SL, diAc C18:1 acidic SL and diAc C18:10 acidic SL. Indeed, also the novel sble deletion strain, for which strain the entire coding sequence was removed (Δsble_full), surprisingly and in contrast to previous observations and reports thus also produces (acetylated) bola SLs. Moreover, fully acetylated bola SLs (tetra acetylated C18:1 bola SLs) are produced in quite abundant amounts. This was completely unexpected as Van Bogaert et al. (2016) stated that the absence of acetyl groups triggers the formation of bola sophorolipids starting from acidic sophorolipids.

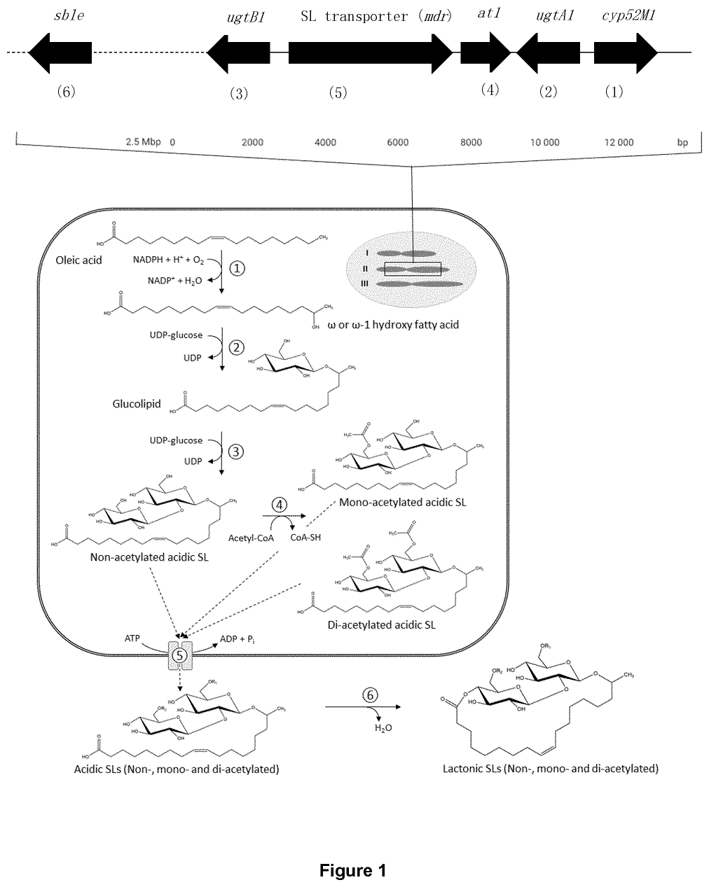

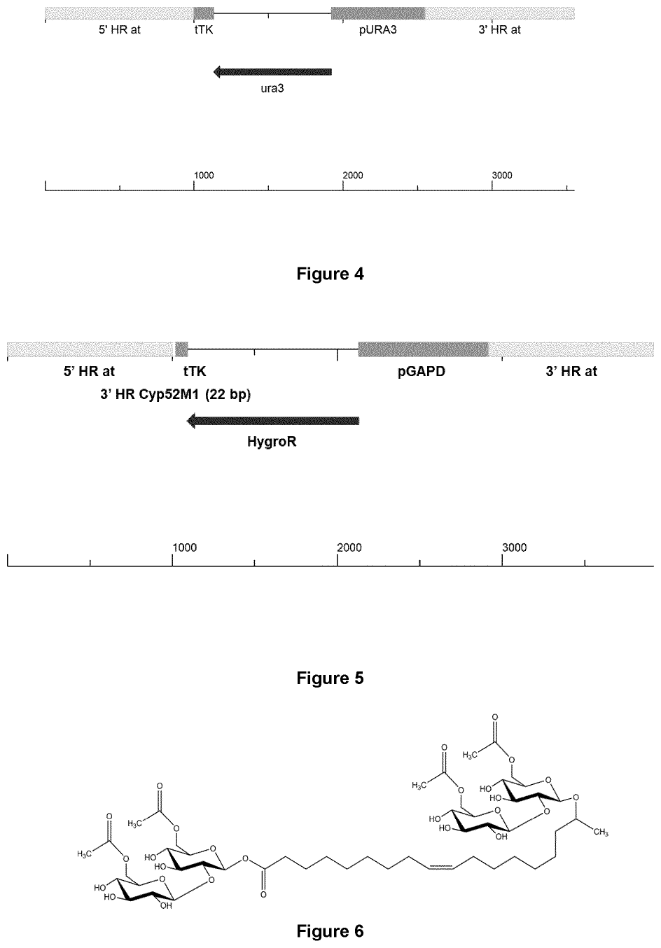

Upon analysis of the novel Δat1_full strain, in which the at1 gene from the SL biosynthetic gene cluster had been completely deleted, similarly as described above for the original strain, indeed again acetylated glycolipid compounds were detected. However, lower acetylation degrees as described above for the Δsble strain were observed. The production spectrum of the Δat1 strain consists mainly of m/z values matching the monoisotopic masses of non-acetylated (nAC) C18:1 bola SL (bola SL), mono-acetylated (mAc) C18:1 bola SL, nAc C18:1 triglucolipids, nAc C16:0 acidic SL, nAc C18:1 acidic SL, nAc C18:0 acidic SL, nAc C18:1 glucolipid, nAc C18:1 L SL and mono-acetylated C18:1 lactonic SL. As the acetyltransferase from the SL biosynthetic gene cluster (FIG. 1, at1) was fully removed in the novel Δat1_full strain as shown in FIG. 4 and acetylated compounds were still observed, this indicates the activity of an unknown acetyltransferase active on SLs.

Lastly, the Δat1Δsble_full S. bombicola strain was generated. In this strain both the sble and the at1 genes/enzymes were fully deleted. The new full Δat1Δsble deletion strain was evaluated as described above and the SL production spectrum was evaluated and found to predominantly consist of m/z values matching the monoisotopic masses of nAc C16:1 bola SL, nAc C16:0 bola SL, nAc C18:1 bola SL, mAc C18:1 bola SL, nAc C18:0 bola SL, nAc C18:1 acidic SL, nAc acidic C18:0 SL and nAc C18:1 glucolipids. Table 4 lists all detected m/z values, corresponding retention times and SL congeners with matching monoisotopic masses.

In general, it is observed that lactonic SL are only observed when no deletion was performed on the sble ORF. Furthermore, the Δat1 strain produces predominantly bola SLs and lactonic SLs with lower acetylation degrees (mAc). While the Δsble strain produces mainly bola SLs with higher acetylation degrees (diAc, triAc and tetraAc). The Δat1Δsble strain predominantly produces bola SLs with lower acetylation degrees (mAc). This indicates that the Sble enzyme has a preference to perform a transesterification reaction on acetylated bola amphiphilic glycolipids.

These findings are in contrast with what is described in the art (Van Bogaert et al. (2016), Van Renthergem et al. (2019), WO 2013/092421 and WO/2021/229017; namely bola sophorolipids can ONLY be produced as completely non-acetylated molecules, because deletion of the at1 gene was described to be required to generate bola sophorolipids. The At1 enzyme was moreover described to be the only enzyme acetylating (bola) glycolipids in S. bombicola (Saerens et al. (2011b), Van Bogaert et al (2016), so the described bola sophorolipids in the art did not contain any acetylgroups.

Deletion of the ugtB1 gene in the Δsble1 strain gave rise to the Δsble1Δugtb1 strain. The production spectrum of the Δsble1Δugtb1 strain consists mainly of m/z values matching the monoisotopic masses of nAc C18:1 bola GL, nAc C18:1 acidic GL, nAc C18:0 acidic GL and mAc C18:1 acidic GL. mAc C18:1 bola GL, nAc C16:0 acidic GL, mAc C18:0 acidic GL and mAc C16:0 GL were found to be present in minor amounts. This finding is also in contrast to what is described in the art, namely that a Δsble1Δugtb1Δat1 strain would be required to produce bola glucolipids and that these bola glucolipids would be expected to be completely non-acetylated. Upon analysis of this strain Δsble1Δugtb1Δat1 also acetylated bola glucolipids were detected. An Δsble1Δugtb1Δat1Δat2Δat3 strain can be used to produce completely non acetylated bola glucolipids (as also described in example 3.

TABLE 1
Deletion cassettes and their respective gene of interest, selection marker, E. coli colony PCR primers and CPEC fragments.
Primers used for CPEC fragment amplification, and the origin of the fragment are listed as well. S. bombicola genome (gDNA).

| Deletion | | | E. coli colony | CPEC | | |
|---|---|---|---|---|---|---|
| cassette | Gene | Selection marker | PCR primers | fragment | Fragment amplification primers | Fragment origin |
| 1 | sble | URA3 | P275, P276 | 1 | oCARBO10279, oCARBO10344 | gDNA |
| | | | | 2 | oCARBO10277, oCARBO10280 | gDNA |
| | | | | 3 | oCARBO10347, oCARBO10278 | gDNA |
| | | | | 4 | oCARBO10346, oCARBO10345 | pJET |
| 2 | at1 | URA3 | P275, P276 | 1 | oCARBO10281, oCARBO10285 | gDNA |
| | | | | 2 | oCARBO10284, oCARBO10288 | gDNA |
| | | | | 3 | oCARBO10286, oCARBO10282 | gDNA |
| | | | | 4 | oCARBO10283, oCARBO10287 | pJET |
| 3 | at1 | Hygromycine | P275, P276 | 1 | oCARBO10281, oCARBO10285 | gDNA |
| | | resistance | | 2 | oCARBO10663, oCARBO10666 | (Van Bogaert et al., 2008) |
| | | | | 3 | oCARBO10664, oCARBO10665 | gDNA |
| | | | | 4 | oCARBO10283, oCARBO10287 | pJET |

TABLE 2
Created strains and their respective deletion cassettes, cassette amplification primers, S. bombicola
PCR primers (5′, 3′ and full overlap), the original strain and the acquired genotype.

| | Deletion | | yeast colony PCR | yeast colony PCR | yeast colony PCR | |
|---|---|---|---|---|---|---|
| Strain | cassette | Cassette amplification primers | primers (5′) | primers (3′) | primers (full overlap) | Original strain |
| Δsble | 1 | oCARBO10349, oCARBO10348 | P1666, P30 | P2458, P1664 | P1666, P1087 | Δsble |
| Δat1 | 2 | oCARBO10350, oCARBO10351 | P465, P410 | P34, P461 | P465, P461 | Δat1 |
| Δsble Δat1 | 3 | oCARBO10350, oCARBO10351 | P465, P1676 | P119, P461 | P465, P461 | Δsble Δat1 |

TABLE 3
Primers used and their sequences

| Primer code | Sequence | Sequence Number |
|---|---|---|
| P30 | AAGGCGGGCTGGAATGCATATCTGAG | SEQ ID No 9 |
| P34 | GATGTCGAATAGCCGGGCTGCTAC | SEQ ID No 10 |
| P119 | ATACCGCTAACCAGAAGGTC | SEQ ID No 11 |
| P275 | CGACTCACTATAGGGAGAGCGGC | SEQ ID No 12 |
| P276 | AAGAACATCGATTTTCCATGGCAG | SEQ ID No 13 |
| P410 | TACCGGAAGGAACCCGCGCTATG | SEQ ID No 14 |
| P461 | CCGCAGTGATCATACCTTAG | SEQ ID No 15 |
| P465 | TGTATGGAGTGAGGAAGGTT | SEQ ID No 16 |
| P1087 | CAGAATATCGCGGGACGCAG | SEQ ID No 17 |
| P1664 | ACCAAGGGGTGATCTCTCGAGATGG | SEQ ID No 18 |
| P1666 | TGCTCGTCTGAGACGGCTTGGG | SEQ ID No 19 |
| P1676 | GAGGCCGTGGTTGGCTTGTATG | SEQ ID No 20 |
| P2458 | CCGGGTATACTAGTGATTTG | SEQ ID No 21 |
| oCARBO10277 | TGGATCCGTTGGGATTCAAATGGTCTCAAAGTGAGGTTGAACCATGATGGCAGTGTTCG | SEQ ID No 22 |
| oCARBO10278 | CTGCCATCATGGTTCAACCTCACTTTGAGACCATTTGAATCCCAACGGATCCACATTG | SEQ ID No 23 |
| oCARBO10279 | GGGTCGTTTGTTCAAATCACTAGTATACCCGGATAGGTTGATGATACACAGCATTCTCG | SEQ ID No 24 |
| oCARBO10280 | GCCGAGAATGCTGTGTATCATCAACCTATCCGGGTATACTAGTGATTTGAACAAACG | SEQ ID No 25 |
| oCARBO10281 | GCTGAGAATATTGTAGGAGATCTTCTAGAAAGATGCTGCAGACAAGTTCCTGCAGCTGTG | SEQ ID No 26 |
| oCARBO10282 | CCAGATCTTCCGGATGGCTCGAGTTTTTCAGCAAGTGCTTTATTCAGGCACGCTACG | SEQ ID No 27 |
| oCARBO10283 | TGCAGGAACTTGTCTGCAGCATCTTTCTAGAAGATCTCCTACAATATTC | SEQ ID No 28 |
| oCARBO10284 | GAAGAAAAGGCTGTCATGAATTTAGTTTACGTGAGGTTGAACCATGATGGCAGTGTTCG | SEQ ID No 29 |
| oCARBO10285 | CGAACACTGCCATCATGGTTCAACCTCACGTAAACTAAATTCATGACAGCCTTTTCTTC | SEQ ID No 30 |
| oCARBO10286 | GTCGTTTGTTCAAATCACTAGTATACCCGGTGAATTCTAGAATGTGAGGTGGAATGAGG | SEQ ID No 31 |
| oCARBO10287 | ATATTCTCACGTAGCGTGCCTGAATAAAGCACTTGCTGAAAAACTCGAGCCATCCGGAAG | SEQ ID No 32 |
| oCARBO10344 | CTTCCGGATGGCTCGAGTTTTTCAGCAAGATCAGACGCATTGGCTGCCTTCTCAG | SEQ ID No 33 |
| oCARBO10345 | CGGGCTGAGAAGGCAGCCAATGCGTCTGATCTTGCTGAAAAACTCGAGCCATCCG | SEQ ID No 34 |
| oCARBO10346 | CACAACACAACGATCGGCAGAGCAGTAATCTTTCTAGAAGATCTCCTACAATATTCTC | SEQ ID No 35 |
| oCARBO10347 | GAATATTGTAGGAGATCTTCTAGAAAGATTACTGCTCTGCCGATCGTTGTGTTGTG | SEQ ID No 36 |
| oCARBO10663 | CTCGATGGAGTGGTAAGCCACTGCCATTGGGTAAACTAAATTCATGACAGCCTTTTCTTC | SEQ ID No 37 |
| oCARBO10664 | AAGAAGAAAAGGCTGTCATGAATTTAGTTTACCCAATGGCAGTGGCTTACCACTCCATCG | SEQ ID No 38 |
| oCARBO10665 | GCCTCATTCCACCTCACATTCTAGAATTCACTTCCATGGGAGGCTAAGAAACG | SEQ ID No 39 |
| oCARBO10666 | AAACGCACGTTTCTTAGCCTCCCATGGAAGTGAATTCTAGAATGTGAGGTGGAATGAG | SEQ ID No 40 |

TABLE 4
Molecular masses determined using UHPLC-HRMS analysis and corresponding
SL congeners in the different S. bombicola strains.

| Molecular | SL Congener ([M − H] match) | | | WT | Δat1 | Δsble | Δat1Δsble |
|---|---|---|---|---|---|---|---|
| Mass | acetylation | hydrophobic tail | type | (n = 3) | (n = 3) | (n = 3) | (n = 3) |
| 918 | non | C16:1 | bola SL | | x | | x |
| 920 | non | C16:0 | bola SL | | x | | x |
| 944 | mono | C16:0 | bola SL | | | | x |
| 946 | non | C18:1 | bola SL | x | x | x | x |
| 988 | mono | C18:1 | bola SL | | x | x | x |
| 784 | non | C18:1 | triglucolipid | | x | x | x |
| 1028 | di | C18:2 | bola SL | x | | x | |
| 596 | non | C16:0 | acidic SL | | x | | x |
| 1030 | di | C18:1 | bola SL | x | x | x | |
| 622 | non | C18:1 | acidic SL | x | x | x | x |
| 1072 | tri | C18:1 | Bola SL | x | | x | |
| 624 | non | C18:0 | acidic SL | x | x | x | x |
| 1114 | tetra | C18:1 | bola SL | | | x | |
| 664 | mono | C18:1 | acidic SL | | x | | |
| 1116 | tetra | C18:0 | bola SL | | | x | |
| 604 | non | C18:1 | lactonic | x | x | | |
| 706 | di | C18:1 | acidic SL | x | | x | |
| 708 | di | C18:0 | acidic SL | x | | x | |
| 606 | non | C18:0 | lactonic | x | x | | |
| 646 | mono | C18:1 | lactonic | x | x | | |
| 688 | di | C18:1 | lactonic | x | | | |
| 690 | di | C18:0 | lactonic | x | | | |

### Example 2

Use of the Sble Enzyme to Perform a Transesterification and/or a Hydrolysis Reaction:

### Material and Methods

### Production of Recombinant Sble

For recombinant Sble (rSble) production, a HAC1 co-expressing strain of P. pastoris (syn. Komagataella phaffii) NRRL-Y-11430 transformed with the pPICZαB_rSbleopt construct which harbours the highest yield of rSble described in De Waele et al. (2018), was utilized in the research. The strain was grown in buffered glycerol-complex medium (BMGY) in 3 L baffled shake flasks containing 500 ml medium for 48 h at 28° C., 250 rpm. Then, the induction was performed in buffered-methanol complex (BMMY) medium for 48 h at 16° C., 250 rpm. Every 12 h, 1% methanol was added for continuous stimulation of protein production. Both BMGY and BMMY consist of 1% (w/v) yeast extract (Lab M), 2% (w/v) peptone (BD), 100 mM phosphate buffer (Chem-Lab) at pH 6.0 and 1.34% (w/v) yeast nitrogen base (YNB, Formedium) with 1% (v/v) glycerol (Chem-Lab) or 1% (v/v) methanol (Chem-Lab) as sole carbon source respectively. Finally, the cultures containing the produced rSble were centrifuged (5000 g, 10 min) to collect the supernatant for protein purification.

### Purification Recombinant Sble

For purification of rSble, a two-step purification strategy was utilized by following the protocol described in De Waele et al. (2018). In brief, for the first step, purification was done on an ÄKTA Purifier system (GE Healthcare). Before sample loading, 0.01% (w/v) reduced glutathione (Sigma-Aldrich) and 2 mM (final concentration) of magnesium sulfate (Sigma-Aldrich) were added in the supernatant, after which the pH was adjusted to 7.5. After removing precipitation by filtering the sample through a Steritop® Filter Unit (EMD Millipore) or VacuCap® (VWR) with a pore size of 0.22 μm, the filtrate was subsequently loaded on a HisTrap™ HP column (5 ml, Cytiva) previously equilibrated with binding buffer of 50 mM Na₂HPO4 (Chem-Lab), pH 7.5, 500 mM NaCl (Chem-Lab) at a flow rate of 5 mL/min. Following sample loading, the column was washed with binding buffer until the UV (280 nm) absorbance reached a steady baseline. Then, a stepwise elution was performed via 20- and 200 mM imidazole (Chem-Lab) in binding buffer. The 2 eluted fractions were mixed and immediately desalted via a buffer exchange using 25 mM Tris-HCl (Sigma-Aldrich), pH 7.5, 150 mM NaCl and Amicon® Ultra-15 centrifugal filter devices (Merck) with a 10 kDa cut-off and eventually concentrated to 1 mL. In the second step, the 1 mL concentrated IMAC fraction was injected onto a HiLoad® 16/600 Superdex® 200 pg column (GE Healthcare) equilibrated with the desalting buffer (25 mM Tris-HCl, pH 7.5, 150 mM NaCl) and eluted with the same buffer. The fractions containing rSble were concentrated to 1.0 ml using the Amicon® Ultra-15 centrifugal filter devices (Merck) with a 10 kDa cut-off. The concentration of rSble was determined using the Thermo Scientific™ Coomassie (Bradford) Protein Assay Kit and using the Bio-Rad Microplate Reader model 680. The protein was stored at −80° C. for further catalytic experiments.

Evaluation of the Catalytic Property of rSble

An HPLC-based activity assay was followed as described by De Waele et al. (2018) with some adaptations. In brief, 2 μg of purified rSble was added to 500 μl of reaction buffer, containing 5 mM of acidic SLs or bola SLs provided by INBIO and 50 mM sodium citrate (Merck) at pH 3.5. The mixture was incubated for 1 h at 30° C. and 1400 rpm after which reaction was stopped using 1500 μl 100% (v/v) ethanol (Chem-Lab). After concentrating the sample using a SpeedVac vacuum centrifuge (Thermo Savant, Holbrook, NY) to 250 μl, 100 μl of the samples were analyzed using HPLC coupled with an UV detector. The reaction in which rSble was replaced by the same amount of the buffer (25 mM Tris, 150 mM NaCl, pH7.5) used for protein purification was used as negative control in the assay.

### HPLC and MALDI-TOF MS Analysis of Sophorolipids

Samples of acidic SLs from the catalytic assay were analyzed by HPLC on an Ettan™ LC system (GE Healthcare) using a ZORBAX Eclipse Plus C18 Rapid Resolution 4.6 mm×100 mm column (Agilent) and UV absorption detection (280 nm, GE Healthcare). A gradient of two eluents, an aqueous solution and acetonitrile (ACN), had to be used to separate the components. The gradient started at 30% ACN and linearly increased to 50% in 15 min, after that the gradient increased linearly from 50% ACN till 60% in 10 min. The mixture was kept in this way for 10 min and was then brought back to 30% ACN in 0.1 min. A flow rate of 0.6 mL/min was applied.

Samples of bola SLs from the catalytic assay were analyzed by another HPLC analytic method on the same LC system using a Brownlee Spheri-5 RP-18 Cartridge Column-220 mm×2.1 mm (Perkin Elmer®) and UV absorption detection (280 nm, GE Healthcare). A gradient of the two eluents, aqueous solution and acetonitrile (ACN), was used to separate the components. The gradient started at 30% ACN and linearly increased to 50% in 15 min, after that the gradient increased linearly from 50% ACN till 80% in 30 min. The mixture was kept in this way for 5 min and was then brought back to 30% ACN in 1 min. A flow rate of 0.15 mL/min was applied.

The fractions of significantly changed peaks before and after reaction were collected and the corresponding compound was identified via Matrix-Assisted Laser Desorption Ionization Time-of-Flight Mass Spectrometry (MALDI-TOF MS). The collected fractions were firstly dried under SpeedVac vacuum centrifuge (Thermo Savant, Holbrook, NY) and the dried compounds were then resuspended in 12 μl of 50% ACN (BioSolve)/0.1% trifluoroacetic acid (TFA, Sigma-Aldrich) solution. 1 μl of resuspended compound, mixed with a saturated α-cyano-4-hydroxycinnamic acid solution in a 1:1 ratio was spotted onto an Opti-TOF 384 Well MALDI Plate Insert for MALDI-TOF MS analysis with the MALDI TOF/TOF 4800 Plus (ABSciex).

Additionally, to verify the conversion of bolaSLs to lactonic SLs by rSble, LC-MS analysis was performed. SL samples (dissolved to 1 mg/ml in ethanol) were separated on an Agilent 1100 series HPLC equipped with a quaternary pump and DAD detector, using a Phenomenex Kinetex C18 150×4.6 mm 5μ solid core type column at 35° C., flow rate 1.5 ml/min. A gradient ranging from 20% to 80% acetonitrile in 30 min with 0.1% formic acid was used to separate the products. The HPLC system was coupled to an Agilent G1956B single quadrupole MS detector equipped with an ESI ionization source. The mass spectrometer was set to scan the mass-to-charge range of 600-1200 amu.

### Results

Activity Assay of rSble Using Acetylated and Non-Acetylated Acidic SLs and Crude SLs

The unexpected finding bola SLs in Δsble strains raised questions about the actual substrates of Sble. Therefore, activity tests were performed on different SL samples. First, the activity of Sble, using the recombinantly produced enzyme rSble, towards acidic SLs was tested. The samples used are (1) di-acetylated acidic SLs (C18:1) ω (2) di-acetylated acidic SLs (C18:1) mix ω and ω-1 and (3) non-acetylated acidic SLs (C18:1) mix ω and ω-1.

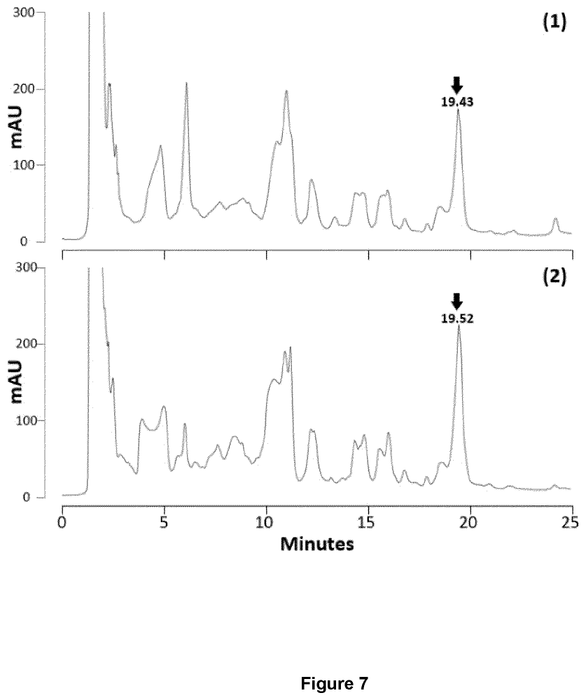

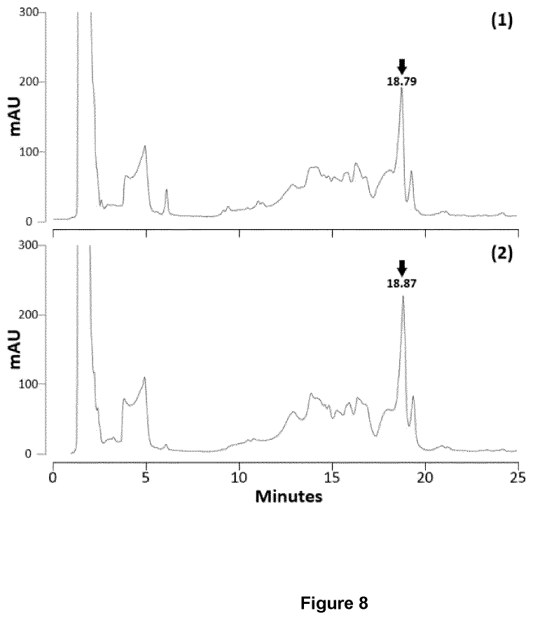

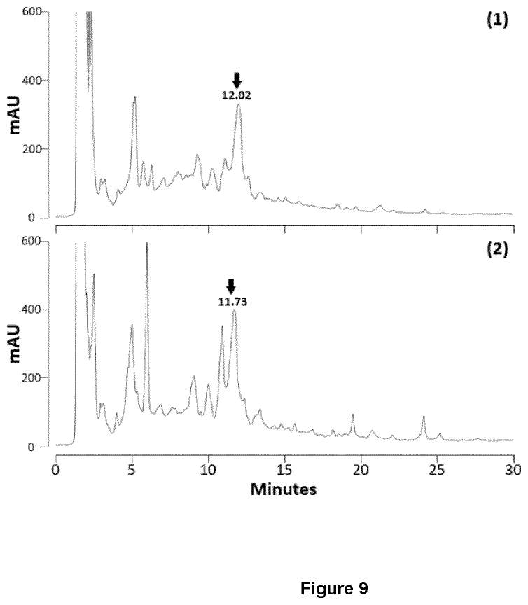

The activity of rSble for the lactonization of the three types of acidic SLs was analyzed using an HPLC-based activity assay following the protocol adapted from Ciesielska et al. (2016) (see methods). A negative control experiment was prepared by adding the buffer without the addition of enzyme. The results showed that no corresponding lactonic SLs were detected after reaction of any of the three acidic SLs (FIGS. 7-9). Indeed, lactonic SLs are expected to elute after 36 and 41 min for the mono-acetylated and di-acetylated lactonic SLs, respectively, (verified by a control experiment, data not shown), and in none of the chromatograms (the occurrence of) such peaks was observed.

As this was a surprising result, the reaction time and concentration of enzyme were increased to investigate whether this was due to a low E: S ratio or slower reaction. Instead of 2 μg, 10 μg of purified rSble was added to the reaction mixture after which the mixture was incubated at 30° C. and 1400 rpm for 2 h. The treatment of reaction mixture and the sample analysis were the same as described above. Again, no lactonic SLs were detected after the reaction indicating that the reaction time and enzyme concentration are not the crucial parameters in view of lactonization of acidic SLs. Additionally, all the samples mentioned previously were analyzed using MALDI-TOF/MS to analyze the possible formation of polymers of di-acetylated acidic SLs. However, inspection of the mass spectra at higher m/Z rate did not provide any indication that polymerization occurred in the reaction.

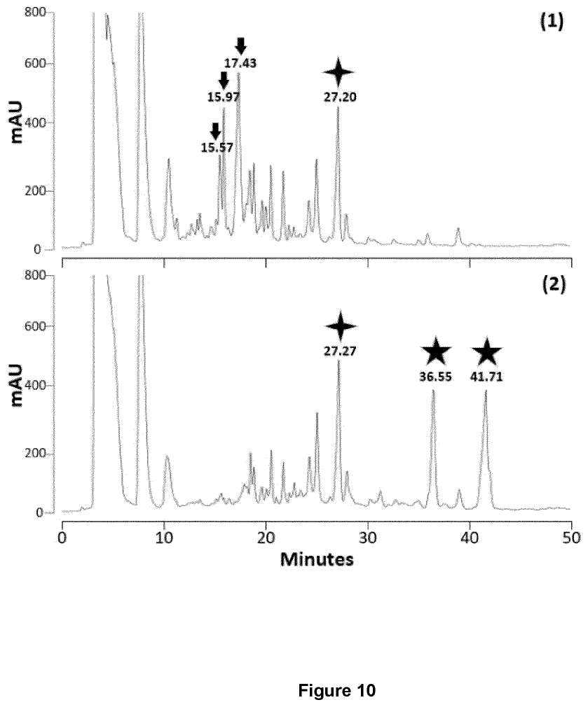

rSble was thus surprisingly not able to convert the three provided acidic SLs into lactonic SL. We returned to an activity test using the original crude SL mixture that was used by Ciesielska et al. (2016) and was used during further investigations to test activity of rSble. This mixture was obtained from the Δsble strain described by (Ciesielska et al., 2014) and not purified/extracted. This crude SLs mixture was always expected to only contain acidic SLs based on the data in the art. However, as described above, the Δsble strain was surprisingly found to produce a mixture of bola SLs and acidic SLs, both in acetylated and non-acetylated form. Indeed, when comparing the HPLC chromatogram of this old SL mixture with the new samples of acidic SLs with high homogeneity, it became clear that additional peaks/compounds (with retention (RT) at 15.5 min, 16.0 min and 17.4 min, (indicated with arrows)) are indeed present in this old crude sample that was used for initial Sble activity assays (FIG. 10). MALDI-TOF MS was performed to investigate the identity of these compounds and fractions collected from the three peaks (RT at 15.5 min, 16.0 min and 17.4 min indicated with an arrow in FIG. 10 above) were analyzed. The mass spectra showed a compound with m/z of 988 (for the peak at RT of 15.5 min), which corresponds to mono-acetylated bola SLs (C18:1). Also, di-acetylated (C18:2) and di-acetylated (C18:1) bola SLs were detected with MW of 1028 and 1030 respectively at RTs of 16.0 min and 17.4 min respectively.

And indeed, exactly these compounds disappear after incubation of this sample (see FIG. 10 panel 2) with rSble giving rise to the formation of di-acetylated lactonic SLs (FIG. 10 below indicated with stars), whereas peaks corresponding to acidic SLs hardly diminish in intensity.

Activity Assay of rSble Using Acetylated and Non-Acetylated Bola SLs

Based on the observation that upon incubating Sble with a mixture of bola SLs and acidic SLs, lactonized SLs are obtained, in contrast to the assays with acidic SLs alone (see above), it was argued that in fact bola amphiphilic glycolipids unexpectedly might be the actual substrate of the Sble enzyme, which then would catalyze a transesterification reaction rather than a lactonizing esterification reaction. In order to confirm this, two types of bola SLs were tested of which the main compounds are: (1) tri-acetylated bola SLs and di-acetylated bola SLs in an approximately 1:1 ratio (code: INV-113) and (2) non-acetylated (and minor amounts of mono-acetylated) bola SLs (code: INV_22).

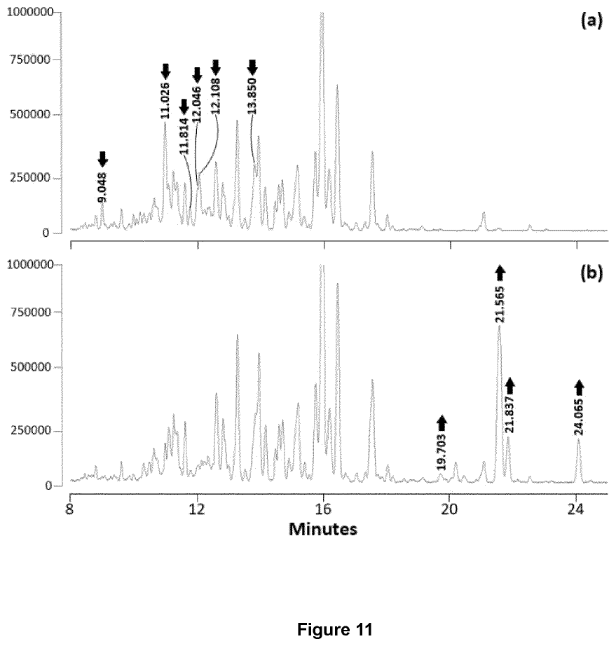

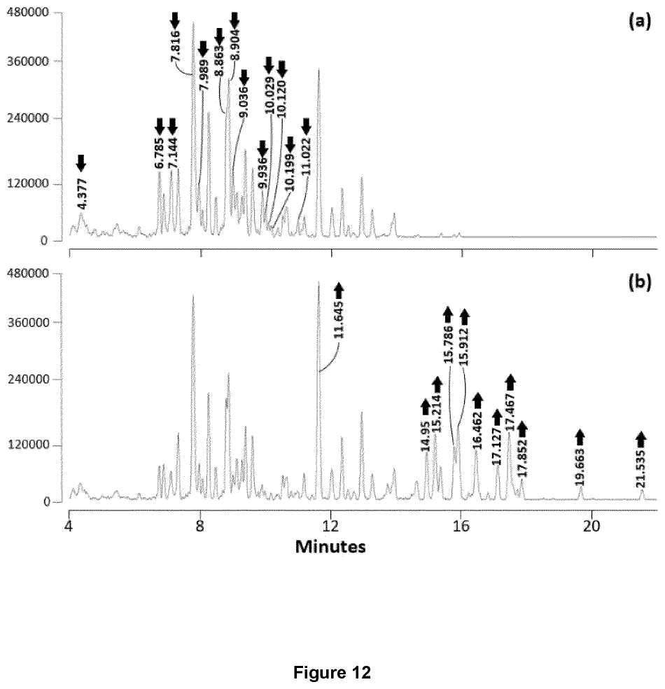

The activity of rSble for the transesterification of the two bola SLs samples to produce lactonic SLs was analyzed using HPLC and MALDI-TOF and LC-MS analysis based on negative spray analysis was performed for identification purposes. For acetylated bola SLs, our data showed that, compared with the negative control (FIG. 11 panel a), four lactonic SLs products (FIG. 11, panel b, Table 5) were formed, in which di-acetylated lactonic SLs (C18:1) are the most abundant product (RT 21,565 minutes in FIG. 11, panel b). The peaks corresponding to the main bola SLs (FIG. 11, panel a, Table 5) disappeared or decreased significantly after the reaction. This was also the case for the sample of mainly non acetylated bola SLs present in the other sample of bola SLs (INV_22) (FIG. 12, Table 6). The results of the activity assays thus further confirms that Sble converts acetylated bola SLs (acetylation degree mono-, di-, tri- and tetra- to form the corresponding Lactonic SLs). All peaks that decreased in intensity after reaction with rSble corresponded to bola SLs, whereas peaks corresponding to acidic SLs remained unchanged after reaction.

TABLE 5
Overview of bola SLs congeners in a sample with higher
acetylation degree that displayed the largest change
in intensity after incubation with rSble and overview
of lactonic SLs appearing as shown in FIG. 11.

| | |
|---|---|
| Molecular Mass | Type |

Decreased bola SLs

| | |
|---|---|
| 988 | mono-Ac bola, C18:1 |
| 1030 | di-Ac bola SL, C18:1 |
| 1072 | tri-Ac bola SL, C18:1 |
| 1032 | di-Ac bola SL, C18:0 |
| 1072 | tri-Ac bola SL, C18:1 |
| 1114 | tetra-Ac bola SL, C18:1 |

Produced lactonic SLs

| | |
|---|---|
| 648 | mono-Ac lactonic SL, C18:0 |
| 688 | di-Ac lactonic SL, C18:1 |
| 688 | di-Ac lactonic SL, C18:1 |
| 690 | di-Ac lactonic SL, C18:0 |

For the sample of bola SLs containing mainly non- and mono-acetylated bola SLs, after the same process and identification of the significantly changed peaks, only minor amounts of lactonic SLs were produced by rSble, although the bola SLs decreased significantly (Table 6). Herein, the peak corresponding to non-acetylated acidic SLs, showed a significant accumulation demonstrating hydrolysis of the ester bond of (non-acetylated) bola SLs, indicating that the enzyme has an additional hydrolysis activity for substrates with low to no acetylation degree.

TABLE 6
Overview of bola SLs in a sample with lower acetylation degree that
displayed the largest change in intensity after incubation with
rSble and overview of lactonic SLs appearing as shown in FIG. 12.

| | |
|---|---|
| Molecular mass | Type |

Significantly decreased bola SLs

| | |
|---|---|
| 962 | mono-Ac bola SL, C16:0 |
| 920 | non-Ac bola SL, C16:0 |
| 920 | non-Ac bola SL, C16:0 |
| 946 | non-Ac bola SL, C18:1 |
| 960 | mono-Ac bola SL, C16:1 |
| 948 | non-Ac bola SL, C18:0 |
| 988 | mono-Ac bola SL, C18:1 |
| 988 | mono-Ac bola SL, C18:1 |
| 990 | mono-Ac bola SL, C18:0 |
| 990 | mono-Ac bola SL, C18:0 |
| 1030 | di-Ac bola SL, C18:1 |
| 1028 | di-Ac bola SL, C18:2 |
| 1030 | di-Ac bola SL, C18:1 |

Produced lactonic SLs

| | |
|---|---|
| 604 | non-Ac lactonic SL, C18:1 |
| 604 | non-Ac lactonic SL, C18:1 |
| 648 | mono-Ac lactonic SL, C18:0 |
| 688 | di-Ac lactonic SL, C18:1 |

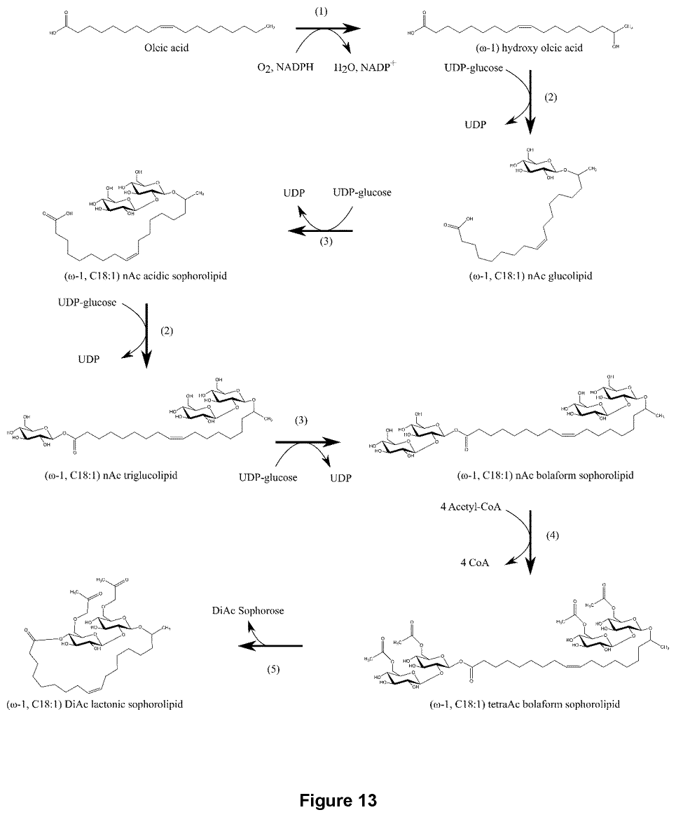

A depiction of the adapted sophorolipid biosynthesis based on these surprising findings is shown in FIG. 13. The biosynthesis of lactonic SLs by S. bombicola as it has been always described and depicted in the art as shown in FIG. 1 i.e. an internal esterification reaction of acidic SLs giving rise to lactonic SLs is thus to be revisited. It was thus found that the biosynthesis of lactonic SLs is instead the result of a transesterification reaction of bola sophorolipids into lactonic sophorolipids. In FIG. 13, the formation and conversion of tetra-acetylated bola SLs is shown, but as shown above, also bola SLs with a lower acetylation degree are converted into non-, mono- and di-acetylated lactonic and/or non-, mono- and di-acetylated acidic SLs.

Activity Assay of rSble Using Methylesters and Saccharides.

To confirm that the SBLE enzyme is capable of performing a transesterification reaction on other substrates, the enzyme was tested for its capacity to transfer a disaccharide on a fatty acid methyl ester. Sophorose was used as the acyl acceptor, whereas methylstearate and methyllaurate were used as the acyl donors (in two separate experiments). The components were mixed in a 1:2 ratio (0.006 mmol:0.012 mmol) in a total volume of 1 ml to which 1 mg/ml of SBLE enzyme was added. The reaction mixture was incubated at 30 degrees for 24 h under agitation. At different time points, samples were taken and analysed through thin layer chromatography and finally LC-MS as described above. For both experiments the appearance of a new compound was evident upon these experiments and the retention time was in the range of that of glycolipids. No appearance of any new compound was observed for the blanc reactions. LC-MS analysis gave evidence of a the appearance of a compound with a mass of 524.6 which corresponds to sophoryl laurate and a mass of 608.8 which corresponds to sophoryl stearate respectively in the reaction mixtures supplied with the SBLE enzyme. This confirms the transesterification activity of the Sble enzyme on other substrates then bola glycolipids.

### Example 3

### Production of Non-Acetylated (Bola) Glycolipids.

### Material and Methods

### Strains and Cultivation Methods

Cloning experiments and plasmid maintenance were performed with Escherichia coli top 10 cells. E. coli cells were grown in Luria-Broth medium (37° C., 10 g/l trypton, 5 g/l yeast extract, 5 g/l sodium chloride and if required 15 g/l agar; Sigma-Aldrich) supplemented with 100 mg/L ampicillin (LB-amp; MP Biomedicals) when applicable. Wild type S. bombicola (WT; ATCC 22214) and an URA3 auxotrophic mutant strain (PT36) were used during this study (Lodens et al., 2018) to serve as base strains to generate a set of novel strains described below. Solid synthetic dextrose with complete supplement mixture without uracil (6.7 g/L Yeast nitrogen base without amino acids (Sigma-Aldrich), 20 g/L glucose (Cargill), 20 g/L agar Noble (Difco), 0.77 g/L complete supplement mixture without uracil (MP biomedicals)) and yeast extract peptone dextrose supplemented with hygromycin (20 g/L glucose (cargill), yeast extract (DSM), 20 g/L bactopepton (BD biosciences), agar (Biokar Diagnostics) were used for selection for positive deletion mutants after transformation with a URA3 auxotrophic marker.

For the glycolipid production experiments, the production medium as described by (Lang et al., 2000) was used. Precultures (5 ml) were inoculated from cryovials (1%) and incubated for 48 h (30° C., 200 rpm). Subsequently, shake flasks (n=3) containing 100 mL production medium were inoculated (1%) from precultures. Shake flasks were incubated for 240 h (30° C., 200 rpm). 37.5 g/L oleic acid (Sigma-Aldrich) was supplemented after 48 h of cultivation.

### Analytical Techniques

Production samples were analysed by UPLC-HRMS (Thermo Scientific™ Exactive™ Plus Orbitrap Mass Spectrometer). Products were separated by UPLC according to (Van Renterghem et al., 2018). Sample preparation was performed on SF broth samples. Firstly, 70% EtOH (3:1, v/v) was added to the sample and vigorously vortexed for 5 min. Subsequently, a centrifugation step was performed (5 min, 14 000 rpm) on which the supernatants was filtered through a PES filter (0.2 μm, sartorius).

### Molecular Methods

Circular polymerase extension cloning (CPEC) pieces and the linear deletion cassettes were amplified with Primestar® GXL according to the manufacturer's instructions. Colony PCR was performed on E. coli and S. bombicola according to (De Graeve et al., 2019). S. bombicola colony PCRs were performed to analyse the 5′, 3′ and full overlap of the integration of the deletion cassette in the genome. CPEC was performed with Q5® Hifi DNA polymerase according to the manufacturer's instructions and as described in (Quan and Tian, 2009). CPEC assembly products and linear deletion cassettes were transformed via electroporation according to (De Graeve et al., 2019) into E. coli and S. bombicola, respectively. Sequencing of CPEC assembled plasmids was performed by Macrogen inc.

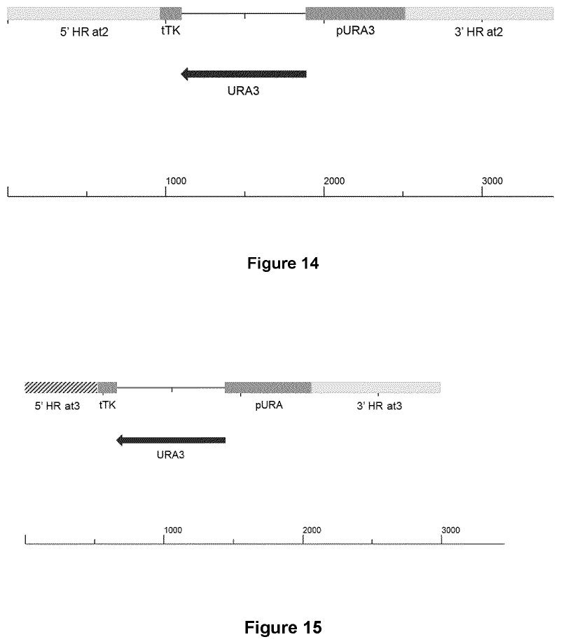

Linear deletion cassettes were generated from vector backbones cloned and maintained in E. coli, based and cloning steps are described below. Two deletion cassettes were constructed for subsequent gene deletion in S. bombicola (FIG. 14-15, Table 7). Genetic elements originate from the S. bombicola genome except the terminator of the Herpes simplex virus tyrosine kinase (tTK) terminator that were used as described by (Van Bogaert et al., 2008). In order to construct the deletion cassettes, fragments were first amplified and assembled with the aid of circular polymerase extension cloning (CPEC) plasmid assembly on a on the pGEM-T (Promega) and pJET (Thermo Fisher) vectors (Quan and Tian, 2009). These plasmids were transformed into E. coli top 10 cells and positive colonies were selected from LB-amp and verified by colony PCR and subsequent DNA sequencing. Primers used for amplification of fragments, amplification origin of fragments and primers used for E. coli colony PCR are listed in Table 8.

TABLE 7
Deletion cassettes and their respective gene of interest, selection marker, E. coli
colony PCR primers and CPEC fragments. Primers used for CPEC fragment amplification.

| Deletion | Gene of | S. bombicola | E. coli colony | CPEC | Fragment |
|---|---|---|---|---|---|
| cassette | interest | selection marker | PCR primers | fragment | amplification primers |
| 4 | at1 | /(used for | P275, P276 | 1 | oCARBO12353, oCARBO10282 |
| | | URA3 recovery) | | 2 | oCARBO10281, oCARBO12352 |
| | | | | 3 | oCARBO10283, oCARBO10287 |
| 5 | aT2 | URA3 | oCARBO10027, | 1 | oCARBO12379, oCARBO12385 |
| | | | oCARBO10028 | 2 | P1014, P2458 |
| | | | | 3 | oCARBO12386, oCARBO12380 |
| 7 | aT3 | URA3 | oCARBO10027, | 1 | oCARBO12395, oCARBO12389 |
| | | | oCARBO10028 | 2 | P1014, P2458 |
| | | | | 3 | oCARBO12477, oCARBO12646 |

TABLE 8
Primers used and their sequences

| Primer code | Sequence | Sequence |
|---|---|---|
| oCARBO12352 | GCCTCATTCCACCTCACATTCTAGAATTCAGTAAACTAAATTCATGAC | SEQ ID No 41 |
| oCARBO12353 | GGCTGTCATGAATTTAGTTTACTGAATTCTAGAATGTGAG | SEQ ID No 42 |
| oCARBO12379 | TGTCGTCGGTCGGTGAGTAG | SEQ ID No 43 |
| oCARBO12380 | ATAGAATCCACCGGCGTTG | SEQ ID No 44 |
| oCARBO12381 | ATGATAGGAGTTTTAACAACGCCGGTGGATTCTATGGCCGCCTGCAGGTCGAC | SEQ ID No 45 |
| oCARBO12382 | TTGGGAGAGCGACTACTCACCGACCGACGACAGCCCAATTCGCCCTATAGTG | SEQ ID No 46 |
| oCARBO12383 | CCGAACTCGTTAAGGTCAATCCACTCAGCCAACACTGCAACGCATTTG | SEQ ID No 47 |
| oCARBO12384 | CGTTGCAGTGTTGGCTGAGTGGATTGACCTTAACGAGTTC | SEQ ID No 48 |
| oCARBO12385 | GATCAGAATTCGAACACTGCCATCATGGTTCAACGATTGACCTTAACGAGTTC | SEQ ID No 49 |
| oCARBO12386 | GTTGGGTCGTTTGTTCAAATCACTAGTATACCCGGCACTCAGCCAACACTGCAAC | SEQ ID No 50 |
| oCARBO12387 | CAGAACCTGCAAGGAGAAC | SEQ ID No 51 |
| oCARBO12388 | ACGGACGTTGCGTTCATGC | SEQ ID No 52 |
| oCARBO12389 | TCCATAGGTCTCACCCATTC | SEQ ID No 53 |
| oCARBO12390 | ACCGGGTTAGCTCTGTTGC | SEQ ID No 54 |
| oCARBO12392 | CCGATGAGGAAGGAGAATGGGTGAGACCTATGGAGCCCAATTCGCCCTATAGTG | SEQ ID No 55 |
| oCARBO12395 | GATCAGAATTCGAACACTGCCATCATGGTTCAACGAATTCCGCTTAGTCTCGTAC | SEQ ID No 56 |
| oCARBO12397 | CTAGCGCTGCCAATGTAAC | SEQ ID No 57 |
| oCARBO12477 | GGTCGTTTGTTCAAATCACTAGTATACCCGGCAGCTCTGTGGCGAAATAC | SEQ ID No 58 |
| oCARBO12646 | ATTCTGGGGAGGATGAATC | SEQ ID No 59 |
| oCARBO12647 | CTGCCGCAGAGGATTCATCCTCCCCAGAATGGCCGCCTGCAGGTCGAC | SEQ ID No 60 |

### Results

Evaluation of Activity of At2 and At3 Enzymes in S. bombicola

Biosurfactant production experiments as described under materials and methods with the S. bombicola strains described in the art: Δat1Δsble strain (Van Bogaert et al., 2016 and WO2015/028278) Δat1 Saerens et al. (2011b) resulted in an unexpected observation in contradiction with the art i.e. the surprising detection of acetylated (bola) sophorolipids up to an acetylation degree of 2 (mainly acetylation degree of 1) in the samples from the experiments with the Δat1 and Δat1 Δsble strains and thus in contrast to these previous observations and reports. This observation was in contradiction with the art and unexpected as the Δat1 Δsble strain and Δat1 had been described not to produce any acetylated (bola) SLs, due to mutation of the at1 gene present in the SL biosynthetic gene cluster, and the corresponding At1 enzyme (Genbank accession number HQ670751) had been described as the (only) enzyme responsible for acetylation of sophorolipids. As already mentioned in example 1, this points to the unexpected activity of other unknown acetyltransferases active on glycolipids in S. bombicola. BlastP analysis was performed using the acetyltransferase protein sequence from the SL biosynthetic gene cluster (SEQ N° 3-SEQ N° 4) against all translated ORFs from the S. bombicola genome and a large number of 71 hits was obtained. Two were selected for further investigation and the respective protein and gene sequences are shown in SEQ N° 5-SEQ N° 8. When performing a DELTA-BLAST on NCBI, these proteins show best homology with maltose- and galactoside O-acetyltransferases mostly of bacterial origin.

Creation and Evaluation of Novel S. bombicola Strains

The deletion cassettes described under materials and methods and shown FIGS. 14-15 were amplified from their respective plasmids and used for transformation into S. bombicola strains. After successful verification, the deletion cassettes were used to generate the strains listed in Table 8.

TABLE 8
Created strains and their respective deletion cassettes, cassette amplification
primers, S. bombicola PCR primers (5′, 3′ and full overlap).

| | Cassette | yeast | yeast | yeast colony |
|---|---|---|---|---|
| | amplification | colony PCR | colony PCR | PCR primers |
| Strain | primers | primers (5′) | primers (3′) | (full overlap) |
| Δsble | oCARBO10349, | P1666,P30 | P2458, P1664 | P1666, P1087 |
| | oCARBO10348 | | | |
| Δat1 | oCARBO10350, | P465, P410 | P34, P461 | P465, P461 |
| | oCARBO10351 | | | |
| Δsble Δat1 | oCARBO10350, | P465, P1676 | P119, P461 | P465, P461 |
| | oCARBO10351 | | | |
| Δat1 Δat2 | oCARBO12379, | oCARBO12388, | P1555, | oCARBO12387, |
| | oCARBO12380 | P1087 | oCARBO12387 | oCARBO12388 |
| Δat1 Δat3 | oCARBO12389, | oCARBO12390, | P1555, | oCARBO12390, |
| | oCARBO12646 | P1087 | oCARBO12397 | oCARBO12397 |
| Δat1 Δat2 | oCARBO12389, | oCARBO12390, | P1555, | oCARBO12390, |
| Δat3 | oCARBO12646 | P1087 | oCARBO12397 | oCARBO12397 |
| Δsble Δat1 | oCARBO12379, | oCARBO12388, | P1555, | oCARBO12387, |
| Δat2 | oCARBO12380 | P1087 | oCARBO12387 | oCARBO12388 |
| Δsble Δat1 | oCARBO12389, | oCARBO12390, | P1555, | oCARBO12390, |
| Δat3 | oCARBO12646 | P1087 | oCARBO12397 | oCARBO12397 |
| Δsble Δat1 | oCARBO12389, | oCARBO12390, | P1555, | oCARBO12390, |
| Δat2 Δat3 | oCARBO12646 | P1087 | oCARBO12397 | oCARBO12397 |

The newly developed strains were evaluated for their production characteristics in shake flask (SF) experiments together with the S. bombicola wild type (WT) strain. Production samples obtained at 180 h after inoculation were subjected to UHPLC-HRMS analysis. Table 9 lists all detected m/z values, corresponding retention times and SL congeners with matching monoisotopic masses.

TABLE 9
Molecular masses determined by UHPLC-HRMS analysis and corresponding
SL congeners in the strains described in this example.

| | SL Congener ([M − H] match) | | | WT | Δat1 | Δat1 Δat2 | Δat1Δat2 Δαt3 |
|---|---|---|---|---|---|---|---|
| Molecular mass | acetylation | hydrophobic tail | type | (n = 3) | (n = 3) | Aat3 (n = 3) | Δsble (n = 3) |
| 918 | non | C16:1 | bola SL | | x | x | x |
| 920 | non | C16:0 | bola SL | | x | x | x |
| 944 | mono | C16:0 | bola SL | | | | |
| 946 | non | C18:1 | bola SL | x | x | x | x |
| 988 | mono | C18:1 | bola SL | | x | | |
| 784 | non | C18:1 | triglucolipid | | x | x | |
| 1028 | di | C18:2 | bola SL | x | | | |
| 596 | non | C16:0 | acidic SL | | x | x | |
| 1030 | di | C18:1 | bola SL | x | x | | |
| 622 | non | C18:1 | acidic SL | x | x | x | x |
| 1072 | tri | C18:1 | bola SL | x | | | |
| 624 | non | C18:0 | acidic SL | | x | x | x |
| 1114 | tetra | C18:1 | bola SL | x | | | |
| 664 | mono | C18:1 | acidic SL | | x | | |
| 1116 | tetra | C18:0 | bola SL | | | | |
| 604 | non | C18:1 | lactonic | x | x | x | |
| 706 | di | C18:1 | acidic SL | x | | | |
| 708 | di | C18:0 | acidic SL | x | | | |
| 606 | non | C18:0 | lactonic | x | x | x | |
| 646 | mono | C18:1 | lactonic | x | x | | |
| 688 | di | C18:1 | lactonic | x | | | |
| 690 | di | C18:0 | lactonic | x | | | |

The wild type S. bombicola produces predominantly C18:1 di-acetylated (diAc) lactonic SLs (L SLs) as expected. The production spectrum of the Δat1 strain consists mainly of m/z values matching the monoisotopic masses of non-acetylated (nAC) C18:1 bola SL (bola SL), mono-acetylated (mAc) C18:1 bola SL, nAc C18:1 triglucolipids, nAc C16:0 acidic SL, nAc C18:1 acidic SL, nAc C18:0 acidic SL, nAc C18:1 glucolipid, nAc C18:1 L SL and mono-acetylated C18:1 lactonic SL. The Δat1Δsble deletion strain SL production spectrum was found to predominantly consist of m/z values matching the monoisotopic masses of nAc C16:1 bola SL, nAc C16:0 bola SL, nAc C18:1 bola SL, mAc C18:1 bola SL, nAc C18:0 bola SL, nAc C18:1 acidic SL, nAc acidic C18:0 SL and nAc C18:1 glucolipids. These findings are in contrast with what is described in the art (Van Bogaert et al. (2016), Van Renthergem et al. (2019), WO 2013/092421 and WO/2021/229017; namely bola sophorolipids can ONLY be produced as completely non-acetylated molecules, because deletion of the at1 gene was described to be required to generate bola sophorolipids. The At1 enzyme was moreover described to be the only enzyme acetylating (bola) glycolipids in S. bombicola (Saerens et al. (2011b), Van Bogaert et al (2016), so the described bola sophorolipids in the art did not contain any acetylgroups.

The SL production spectrum of the Δat1Δat2Δat3 strain mainly consists of nAc C18:1 bola SL and nAc C18:1 lactonic SLs, but also nAc C18:1 acidic SL. Upon deletion of the sble gene in this last strain, the Δat1Δat2Δat3Δsble strain was obtained as described in materials and methods and it was found that this strain mainly produces non-acetylated bola sophorolipids such as nAc C16:1 bola SL, nAc C16:0 bola SL, nAc C18:1 bola SL, nAc C18:0 bola SL, nAc C18:1 acidic SL and nAc C18:1 glucolipids and that no acetylated SLs/GLs or other acetylated (bola) amphiphilic glycolipids are produced anymore. In these analyses, the clear appearance of nAc C18:1 acidic SL for Δat1Δat2Δat3 compared minor amounts in the strain Δat1Δat2Δat3Δsble is in line with the in vitro data described above: Sble will preferably perform a transesterification reaction for acetylated bola amphiphilic compounds, while a hydrolysis reaction exists alongside the transesterification reaction for non-acetylated bola amphiphilic compounds.

This was unexpected as it was assumed that the acetyltransferase gene present in the SL biosynthetic gene cluster (at1) was solely responsible for acetylation of glycolipids in S. bombicola. We have found and shown here that other previously unknown genes/enzymes (at2/At2 and at3/At3) present in the S. bombicola genome also have this acetylation activity on (bola) sophorolipids and glucolipids, but to a lower extent and with different specificity. Fully non-acetylated glycolipid products are interesting due to the variation in properties of the respective fully non-acetylated glycolipid compounds, but also have a clear benefit compared to acetylated glycolipids i.e. the spontaneous ‘release’ of acetic acid in watery environments upon spontaneous hydrolysis of acetyl groups. This gives rise to an unpleasant odour, which will not be the case for glycolipids derived from strains containing a combination of Δat1Δat2Δat3.

### REFERENCES

Ciesielska, K., Van Bogaert, I. N. A., Chevineau, S., Li, B., Groeneboer, S., Soetaert, W., Van de Peer, Y., Devreese, B. 2014. Exoproteome analysis of Starmerella bombicola results in the discovery of an esterase required for lactonization of sophorolipids. J. Proteom. 98, 159-174. DOI10.1016/j.jprot.2013.12.026
Ciesielska, K., Roelants, S. L. K. W., Van Bogaert, I. N. A., De Waele, S., Vandenberghe, I., Groeneboer, S., Soetaert, W., Devreese, B. 2016. Characterization of a novel enzyme-Starmerella bombicola lactone esterase (Sble)-responsible for sophorolipid lactonization. Appl. Microbiol. Biotechnol. 22, 9529-9541. DOI10.1007/s00253-016-7633-2
Chen, J., Song, X., Zhang, H., Qu, Y., & Miao, J. (2006). Sophorolipid produced from the new yeast strain Wickerhamiella domercqiae induces apoptosis in H7402 human liver cancer cells. Applied Microbiology and Biotechnology, 72(1), 52-59. https://doi.org/10.1007/s00253-005-0243-z
De Graeve, M., Van de Velde, I., Saey, L., Chys, M., Oorts, H., Kahriman, H., Mincke, S., Stevens, C., De Maeseneire, S. L., Roelants, S. L. K. W., Soetaert, W. K. G., 2019. Production of long-chain hydroxy fatty acids by Starmerella bombicola. FEMS Yeast Res. 19. https://doi.org/10.1093/femsyr/foz067
De Waele, S., Vandenberghe, I., Laukens, B., Planckaert, S., Verweire, S. Van Bogaert, I. N. A., Soetaert, W., Devreese, B. and Ciesielska, K. 2018. Optimized expression of the Starmerella bombicola lactone esterase in Pichia pastoris through temperature adaptation, codon-optimization and co-expression with HAC1. Protein Express. Pur. 143, 62-70. DOI10.1016/j.pep.2017.10.016
Gorin, P. A. J., Spencer, J. F. T., & Tulloch, A. P. (1961). Hydroxy Fatty Acid Glycosides of Sophorose from Torulopsis magnoliae. Canadian Journal of Chemistry, 39(4), 846-855. https://doi.org/10.1139/v61-104
Imura, T., Masuda, Y., Minamikawa, H., Fukuoka, T., Konishi, M., Morita, T., Sakai, H., Abe, M., & Kitamoto, D. (2010). Enzymatic Conversion of Diacetylated Sophoroselipid into Acetylated Glucoselipid: Surface-Active Properties of Novel Bola Biosurfactants. Journal of Oleo Science J. Oleo Sci, 59(9), 495-501. https://www.jstage.jst.go.jp/article/jos/59/9/59_9_495/_pdf
Konishi, M., Fukuoka, T., Morita, T., Imura, T., & Kitamoto, D. (2008). Production of new types of sophorolipids by Candida batistae. Journal of Oleo Science, 57(6), 359-369. http://www.ncbi.nlm.nih.gov/pubmed/18469499
Kurtzman, C. P. (2012). Candida kuoi sp. nov., an anamorphic species of the starmerella yeast clade that synthesizes sophorolipids. International Journal of Systematic and Evolutionary Microbiology, 62(9), 2307-2311. https://doi.org/10.1099/ijs.0.039479-0
Kurtzman, C. P., Price, N. P. J., Ray, K. J., & Kuo, T. M. (2010). Production of sophorolipid biosurfactants by multiple species of the Starmerella (Candida) bombicola yeast clade. FEMS Microbiology Letters, 311(2), 140-146. https://doi.org/10.1111/j.1574-6968.2010.02082.x
Lang, S., Brakemeier, A., Heckmann, R., Spockner, S., Rau, U., 2000. Production of native and modified sophorose lipids. Chim. OGGI-CHEMISTRY TODAY 18, 76-79.
Lodens, S., De Graeve, M., Roelants, S. L. K. W., De Maeseneire, S. L., Soetaert, W., 2018. Transformation of an Exotic Yeast Species into a Platform Organism: A Case Study for Engineering Glycolipid Production in the Yeast Starmerella bombicola, in: Braman, J. C. (Ed.), Synthetic Biology: Methods and Protocols. Springer New York, New York, NY, pp. 95-123. https://doi.org/10.1007/978-1-4939-7795-6_5
Lodens, S, Roelants, S. L. K. W., Ciesielska, K., Geys, R., Derynck, E., Maes, K., Pattyn, F., Van Renterghem, L., Mottet, L., Dierickx, S., Vanhaecke, L., Devreese, B., De Maeseneire, S. L. and Soetaert, W. 2019. Unraveling and resolving inefficient glucolipid biosurfactants production through quantitative multiomics analyses of Starmerella bombicola strains. Blotechnol. and Bioeng. 117(2): 453-
Price N P, Ray K J, Vermillion K, Dunlap C A, Kurtzman C P. 2012 Structural characterization of novel sophorolipid biosurfactants from a newly-identified species of Candida yeast. Carbohydr Res 348:33-41.
Quan, J., Tian, J., 2009. Circular polymerase extension cloning of complex gene libraries and pathways. PLOS One 4, e6441.
Roelants, S. L. K. W., Ciesielska, K., De Maeseneire, S. L., Moens, H., Everaert, B., Verweire, S., Denon, Q., Vanlerberghe, B., Van Bogaert, I. N. A., Van der Meeren, P., Devreese, B., & Soetaert, W. (2016). Towards the industrialization of new biosurfactants: Biotechnological opportunities for the lactone esterase gene from Starmerella bombicola. Biotechnology and Bioengineering, 113(3), 550-559. https://doi.org/10.1002/bit.25815
Saerens, K.; Van Bogaert, I.; Soetaert, W. and Vandamme, E. J. (2009). Production of glucolipids and specialty fatty acids from sophorolipids by Penicillium decumbens naringinase: optimization and kinetics. Biotechnology Journal, 4:517-524.
Saerens, K. M. J.; Roelants, S. L. K. W.; Van Bogaert, I. N. A. and Soetaert, W. (2011a). Identification of the UDP-glucosyltransferase gene UGTA1, responsible for the first glucosylation step in the sophorolipid biosynthetic pathway of Candida bombicola ATCC 22214. Fems Yeast Research, 11 (1): 123-132.
Saerens, K. M. J.; Saey, L. and Soetaert, W. (2011b). One-Step Production of Unacetylated Sophorolipids by an Acetyltransferase Negative Candida bombicola. Biotechnology and Bioengineering, 108 (12): 2923-2931.
Saerens, K. M. J.; Zhang, J.; Van Bogaert, I. and Soetaert, W. (2011c). Cloning and functional characterization of the UDP-glucosyltransferase UgtB1 involved in sophorolipid production by Candida bombicola and creation of a glucolipid-producing yeast strain. Yeast, 28:279-292.
Saerens, K. M. J; Van Bogaert, I. N. A.; Soetaert, W. (2015). Characterization of sophorolipid biosynthetic enzymes from Starmerella bombicola. FEMS Yeast Research, 15 (7):
Spencer, J. F. T., Gorin, P. A. J., & Tulloch, A. P. (1970). Torulopsis bombicola sp. n. Antonie van Leeuwenhoek, 36(1), 129-133. https://doi.org/10.1007/BF02069014
Tulloch, A. P., Spencer, J. F. T., & Deinema, M. H. (1968). A new hydroxy fatty acid sophoroside from Candida bogoriensis. Canadian Journal of Chemistry, 46(3), 345-348. https://doi.org/10.1139/v68-057
Van Bogaert, I. N. A., De Maeseneire, S. L., Develter, D., Soetaert, W., Vandamme, E. J., 2008. Cloning and characterization of the glyceraldehyde 3-phosphate dehydrogenase gene of Candida bombicola and use of its promotor. J. Ind. Microbiol. Biotechnol. 35, 1085-1092.
Van Bogaert, I. N. A., Holvoet, K., Roelants, S. L. K. W., Li, B., Lin, Y. C., Van de Peer, Y., & Soetaert, W. (2013). The biosynthetic gene cluster for sophorolipids: A biotechnological interesting biosurfactant produced by Starmerella bombicola. Molecular Microbiology, 88(3), 501-509. https://doi.org/10.1111/mmi.12200
Van Bogaert, I. N. A., Buyst, D., Martins, J. C., Roelants, S. L. K. W., & Soetaert, W. K. (2016). Synthesis of bola biosurfactants by an engineered Starmerella bombicola yeast. Biotechnology and Bioengineering, 113(12), 2644-2651. https://doi.org/10.1002/bit.26032
Van Renterghem, L., Roelants, S. L. K. W., Baccile, N., Uyttersprot, K., Taelman, M. C., Everaert, B., Mincke, S., Ledegen, S., Debrouwer, S., Scholtens, K., Stevens, C., Soetaert, W., 2018. From lab to market: An integrated bioprocess design approach for new-to-nature biosurfactants produced by Starmerella bombicola. Biotechnol. Bioeng. 115, 1195-1206. https://doi.org/10.1002/bit.26539
Van Renterghem, L., Clicque, H., Huyst, A., Roelants, S. L. K. W. and Soetaert, W., 2019. Miniaturization of Starmerella bombicola fermentation for evaluation and increasing (novel) glycolipid production. Appl. Microbiol. Biotechnol. 103(11), 4347-4362. DOI: 10.1007/s00253-019-09766-3

1. A method of producing bola amphiphilic glycolipids, said method comprising culturing a modified yeast strain that comprises a non-functional or dysfunctional Starmerella bombicola lactone esterase (Sble), and/or does not comprise a functional sble gene and/or has a reduced expression of sble compared to a non-modified yeast to produce bola amphiphilic glycolipids.

2. The method according to claim 1, wherein said bola amphiphilic glycolipids are acetylated bola amphiphilic glycolipids.

3. The method according to claim 1, wherein said bola amphiphilic glycolipids are bola sophorolipids.

4. The method according to claim 1, wherein said bola sophorolipids have an acetylation degree of 4.

5. The method according to claim 1, wherein the modified yeast further comprises a non-functional or dysfunctional glucosyltransferase enzyme UgtB1, and/or does not comprise a functional ugtB1 gene and/or has a reduced expression of ugtB1 compared to a non-modified yeast, and wherein said bola amphiphilic glycolipids are bola glucolipids.

6. The method according to claim 1, wherein the modified yeast further comprises a non-functional or dysfunctional acetyl transferase enzyme 1 (At1), and/or does not comprise a functional at1 gene from the SL biosynthetic cluster and/or has a reduced expression of at1 compared to a non-modified yeast and wherein said bola amphiphilic glycolipids have an acetylation degree of 0, 1 or 2.

7. The method according to claim 6, wherein the modified yeast further comprises a non-functional or dysfunctional glycoside O-acetyltransferase enzyme 2 (At2) or a non-functional or dysfunctional glycoside O-acetyltransferase enzyme 3 (At3), and/or does not comprise a functional at2 or at3 gene and/or has a reduced expression of at2 or at3 compared to a non-modified yeast wherein said bola amphiphilic glycolipids have an acetylation degree of 0, 1 or 2.

8. The method according to claim 6, wherein the modified yeast further comprises a non-functional or dysfunctional (At2) and a non-functional or dysfunctional (At3), and/or does not comprise a functional at2 and at3 gene and/or has a reduced expression of at2 and at3 compared to a non-modified yeast to produce non-acetylated bola amphiphilic glycolipids, wherein said bola amphiphilic glycolipids are non-acetylated bola sophorolipids and/or non-acetylated bola glucolipids.

9. An isolated acetyltransferase having an amino acid sequence given by SEQ ID N° 6 or SEQ ID N° 8.

10. A method of producing non-acetylated glycolipids, said method comprising culturing a modified yeast strain that comprises a non-functional or dysfunctional acetyl transferase enzyme 1 (At1), a non-functional or dysfunctional glycoside O-acetyltransferase enzyme 2 (At2) and a non-functional or dysfunctional glycoside O-acetyltransferase enzyme 3 (At3) enzyme and/or that does not contain the at1, at2 and at3 genes and/or wherein the genes encoding for the At1, At2 and At3 enzymes are completely disabled or removed to produce non-acetylated glycolipids.

11. The method according to claim 10, wherein the modified yeast comprises a non-functional or dysfunctional Sble and/or UgtB1 enzyme and/or does not contain a sble and/or ugtB1 gene.

12. The method according to claim 1, wherein the modified yeast strain is a yeast strain selected from the group consisting of Starmerella (Candida) bombicola, Starmerella (Candida) apicola, Starmerella (Candida) batistae, Starmerella (Candida) magnolia, Candida gropengiesseri, Starmerella (Candida) floricola, Candida tropicalis, Candida riodocensis, Starmerella (Candida) stellata, Starmerella (Candida kuoi), Candida tropicalis, Candida sp. NRRL Y-27208, Pseudohyphozyma (Rhodotorula, Candida) bogoriensis sp., Wickerharmiella domericqiae, Candida antarctica, Pseudohyphozyma antarctica, Pseudohyphozyma bogoriensis, Candida lipolytica and a sophorolipid-producing strain of the Starmerella clade.

13. A method of performing a transesterification and/or hydrolysis reaction, the method comprising contacting a suitable substrate with a Sble enzyme.

14. The method according to claim 13 to perform a transesterification and/or hydrolysis reaction on bola amphiphilic glycolipids, wherein bola amphiphilic glycolipids are contacted with the Sble enzyme.

15. The method according to claim 14 for the production of lactonic glycolipids, wherein the Sble enzyme converts said bola amphiphilic glycolipids into lactonic glycolipids, wherein said bola amphiphilic glycolipids are bola sophorolipids and wherein said lactonic glycolipids are lactonic sophorolipids, or, wherein said bola amphiphilic glycolipids are bola glucolipids and wherein said lactonic glycolipids are lactonic glucolipids and wherein glucose and/or sophorose which are non-acetylated and/or acetylated are released.

16. The method according to claim 14 for the production of acidic glycolipids, wherein the Sble enzyme converts said bola amphiphilic glycolipids into acidic glycolipids, wherein said bola amphiphilic glycolipids are bola sophorolipids and wherein said acidic glycolipids are acidic sophorolipids, or, wherein said bola amphiphilic glycolipids are bola glucolipids and wherein said acidic glycolipids are acidic glucolipids and wherein glucose and/or sophorose which are non-acetylated and/or acetylated are released.

17. The method according to claim 15, wherein said bola sophorolipids are tetra-acetylated bola sophorolipids and wherein said lactonic sophorolipids are di-acetylated lactonic sophorolipids and wherein glucose and/or sophorose which are non-acetylated and/or acetylated are released.

18. The method according to claim 15, wherein said bola sophorolipids are non, mono-, di- and/or tri-acetylated bola sophorolipids and wherein said lactonic sophorolipids are non-, mono- and/or di-acetylated lactonic sophorolipids and wherein glucose and/or sophorose which are non-acetylated and/or acetylated are released.

19. The method according to claim 16, wherein said bola sophorolipids are tetra-acetylated bola sophorolipids and wherein said acidic sophorolipid are di-acetylated acidic sophorolipids wherein glucose and/or sophorose which are non-acetylated and/or acetylated are released.

20. The method according to claim 16, wherein said bola sophorolipids are non, mono-, di- and/or tri-acetylated bola sophorolipids and wherein said acidic sophorolipids are non-, mono- and/or di-acetylated acidic sophorolipids and wherein glucose and/or sophorose which are non-acetylated and/or acetylated are released.

21. The method according to claim 10, wherein the modified yeast strain is a yeast strain selected from the group consisting of: Starmerella (Candida) bombicola, Starmerella (Candida) apicola, Starmerella (Candida) batistae, Starmerella (Candida) magnolia, Candida gropengiesseri, Starmerella (Candida) floricola, Candida tropicalis, Candida riodocensis, Starmerella (Candida) stellata, Starmerella (Candida kuoi), Candida tropicalis, Candida sp. NRRL Y-27208, Pseudohyphozyma (Rhodotorula, Candida) bogoriensis sp., Wickerharmiella domericqiae, Candida antarctica, Pseudohyphozyma antarctica, Pseudohyphozyma bogoriensis, Candida lipolytica and a sophorolipid-producing strain of the Starmerella clade.


SEQUENCE LISTING (62 sequences)

SEQ ID NO: 1 - DNA, 1233 bp, Starmerella bombicola
  source 1..1233: mol_type = genomic DNA
atgctggctctgtttttttcgcttgcgcctctactttctcaagctctccctttaggctatactgcggcccccgctgaatcattctatttttggccagagaacatatccagcctccaagctggcgagatttttagaaaacgggaactcttaactctcccagacatctttgactttggccctaatctggaaaaggtcgtacaagtggcttacaaaacccgtctcaccgatggcaatgactcgttttccatcgccagtatctttatccctaagaatccaagcccagaactcaaactttactcttatcagacgtttgaggatgccgtgcagcttgattgtgccccaagctatgctttagaagtgggtaacaagtccagcaactatcttcctgtcactagcaatttatctgccatcagtcgagaacttgagaaaggacgtcactgcattatccctgatcacgagggctatatttcaggattctttgcaggacggcaggagggatatgctggtttagacggaattcgcgctgctcgaaactatctcaatggcaccaacgagaccccaattggtatcttcggatacagtggaggtgcacaagcaacggcctggattgttgatttgcatgacgagtatgctcctgacttgaactttgttggaacagtttctggaggcactttggttgacgcttggggcacttttcagtatatcgactatccgaaggtgtatctaaagggcagcattcttatcatgtatacgggtcttttttcaggttatccagctcaatttgaggtgatttggccatatattgagcctgtaattcaagaaaacatgctactgctacgtttggcgccgaatgattgtaaccaaagcccgatacttcaaggttacaacaattcaatcatggccggtatacatgtggaccttcccgaattccctgcttctaagtacatattccagcacgagtccctccttgccaactacagcgtagtgccagtttccacaccgaagtttcctcgctacatgtaccatggtggatctgatgagttggccaaattgagccttgtcgagcagtatgttgatcaacaatggaataccggcgctaatctcaccttcgtggtgtatccgggtcttcttcatgacgagacggcttaccgtggctttgatgccgcgatggattggcttgatgcccagctcgatagtggataccttccacctgtaaactcaactcatacatga

SEQ ID NO: 2 - protein, 410 aa, Starmerella bombicola
MLALFFSLAPLLSQALPLGYTAAPAESFYFWPENISSLQAGEIFRKRELLTLPDIFDFGPNLEKVVQVAYKTRLTDGNDSFSIASIFIPKNPSPELKLYSYQTFEDAVQLDCAPSYALEVGNKSSNYLPVTSNLSAISRELEKGRHCIIPDHEGYISGFFAGRQEGYAGLDGIRAARNYLNGTNETPIGIFGYSGGAQATAWIVDLHDEYAPDLNFVGTVSGGTLVDAWGTFQYIDYPKVYLKGSILIMYTGLFSGYPAQFEVIWPYIEPVIQENMLLLRLAPNDCNQSPILQGYNNSIMAGIHVDLPEFPASKYIFQHESLLANYSVVPVSTPKFPRYMYHGGSDELAKLSLVEQYVDQQWNTGANLTFVVYPGLLHDETAYRGFDAAMDWLDAQLDSGYLPPVNSTHT

SEQ ID NO: 3 - DNA, 780 bp, Starmerella bombicola
  source 1..780: mol_type = genomic DNA
atggttgtaaactcctcgaaggaccctcaaaacaaaggaatgactcctagaaaagaaattgaccaggaaatggtctcttgggccaaaaaaaacctcaaaaacacccctggcaatgaaaactatgagaagatggtctcaggagttccttacaatccatacgatccagatcttatgtttagagccctggctactagtgagaaagttagggagttcaataccattgcaagtgaaagtcgtacttttgagtcaaatcacgctgcttatatcaagaaggtcgagattctcaaagacacttttggtcaaacaaaggatattgtctggctgaccgctccattctcagttgattttggattcaacatcagcgtaggcgagcacttttacgccaacttcaacgtttgcttcttggactcggctccaataatctttggtgatgaggtgattgtagggcccaatacaacgttcgtgactgcgactcatcctattagccccgagaaacgtgcgaggagaattgtgtatgctcttcctatcaaggtggggaataatgtatggattggtgcgaatgtgactgtcctgccgggtgttacgattggagatggctcaacaattgcggctggtgctgtcgttcgagaagatgttcctcctcgtactgtggtgggaggagtccctgcgcgaatcctcaagcatattccagaggaggatcccgacgaggctgaaggagaggaactggaattccttcttccagttgaaatgaacgtcaataccgctaaccagaaggtctag

SEQ ID NO: 4 - protein, 259 aa, Starmerella bombicola
MVVNSSKDPQNKGMTPRKEIDQEMVSWAKKNLKNTPGNENYEKMVSGVPYNPYDPDLMFRALATSEKVREFNTIASESRTFESNHAAYIKKVEILKDTFGQTKDIVWLTAPFSVDFGFNISVGEHFYANFNVCFLDSAPIIFGDEVIVGPNTTFVTATHPISPEKRARRIVYALPIKVGNNVWIGANVTVLPGVTIGDGSTIAAGAVVREDVPPRTVVGGVPARILKHIPEEDPDEAEGEELEFLLPVEMNVNTANQKV

SEQ ID NO: 5 - DNA, 663 bp, Starmerella bombicola
  source 1..663: mol_type = genomic DNA
atgcctagcggagccccaagaatcgagtacaattgggacctgatcaagtgggctcgcgaaaatttgtcccatttgccggtagatgatgacaactatcaccggatgattagtgggttgccatatgaggcaacccgcacagattattcgcgccatcgaatagagtcccatgaattacttctagaatacttgaatatgaaactgaaggacttcgctacgttggaaaaatataatcaggcgcgagcagatttgctttcaaaggtgtttggctccatgggcacaaactgcttcattgagcaacacctatttgtagattatggttgcaacattaaagtcggcaataacttttatgcgaacaacaaccttacaatgctcgattgctctgtcattgagattggcgacaatgtgttttttggacctaatgtaacaatcactacggcatctcacccgttggaatcgaagcctagggccgaaggggtcgaattcgctttcaatgtcaaaatcggaaacaacgtctggataggttccaacgctgtggtcttgccgggagttaccattggagatgacgtagtcgttgcagctggcgcagtggtcaacaaggatgtgcccccttcagtcgtagtgggcggtgtcccggcgaaaattcttaagcaaatccagaattga

SEQ ID NO: 6 - protein, 220 aa, Starmerella bombicola
MPSGAPRIEYNWDLIKWARENLSHLPVDDDNYHRMISGLPYEATRTDYSRHRIESHELLLEYLNMKLKDFATLEKYNQARADLLSKVFGSMGTNCFIEQHLFVDYGCNIKVGNNFYANNNLTMLDCSVIEIGDNVFFGPNVTITTASHPLESKPRAEGVEFAFNVKIGNNVWIGSNAVVLPGVTIGDDVVVAAGAVVNKDVPPSVVVGGVPAKILKQIQN

SEQ ID NO: 7 - DNA, 747 bp, Starmerella bombicola
  source 1..747: mol_type = genomic DNA
atgttgcctgcaacagaaatcgatagagaactcgtgcaatgggctcgcgaaaatcttccaaacctccctcaaagcacacattatgacaagcagattagtggcatgctgatcaagcccaaatggtcctcaatggttcacgagacaaagatgaaacagctcacaagggactatgacagcatcaatctcaaccatttcagctctgtggcgaaatactttgaggccaggacaagctttatccagaagcatctcctcggcaaaacaggaaagagagtctacctcgaatccccagttcacatcaatcacggatacaatatatcggtaggcgaaaacttctattgcaactttaattgcatatttctcgactggtccataatcagaattggcgacaacgttgcgattggccccaactgtaccttaagttgcattaatcatcccttgagtggcgatgatcgcaaaaatggtgcgggtttatacgctttccctatctttatcgatgacaatgtctggataggggcgaactgtgtgattctttcagggattcatgttgctgaagggtcggttgtcgccgcaggatcggtagtgacaaaaagtgtgccccctcatgttatcgtggctggcaatcctgcgaagatcattgcgaaggcaacagaccgacgacttcgggctgccgcagaggattcatcctccccagaatcttcggacgccgaagagagctacatgttcattaccaagactgcggatccctga

SEQ ID NO: 8 - protein, 248 aa, Starmerella bombicola
MLPATEIDRELVQWARENLPNLPQSTHYDKQISGMLIKPKWSSMVHETKMKQLTRDYDSINLNHFSSVAKYFEARTSFIQKHLLGKTGKRVYLESPVHINHGYNISVGENFYCNFNCIFLDWSIIRIGDNVAIGPNCTLSCINHPLSGDDRKNGAGLYAFPIFIDDNVWIGANCVILSGIHVAEGSVVAAGSVVTKSVPPHVIVAGNPAKIIAKATDRRLRAAAEDSSSPESSDAEESYMFITKTADP

SEQ ID NO: 9 - DNA, 26 bp, synthetic construct
  source 1..26: mol_type = other DNA; note = Primer
aaggcgggctggaatgcatatctgag

SEQ ID NO: 10 - DNA, 24 bp, synthetic construct
  source 1..24: mol_type = other DNA; note = Primer
gatgtcgaatagccgggctgctac

SEQ ID NO: 11 - DNA, 20 bp, synthetic construct
  source 1..20: mol_type = other DNA; note = Primer
ataccgctaaccagaaggtc

SEQ ID NO: 12 - DNA, 23 bp, synthetic construct
  source 1..23: mol_type = other DNA; note = Primer
cgactcactatagggagagcggc

SEQ ID NO: 13 - DNA, 24 bp, synthetic construct
  source 1..24: mol_type = other DNA; note = Primer
aagaacatcgattttccatggcag

SEQ ID NO: 14 - DNA, 23 bp, synthetic construct
  source 1..23: mol_type = other DNA; note = Primer
taccggaaggaacccgcgctatg

SEQ ID NO: 15 - DNA, 20 bp, synthetic construct
  source 1..20: mol_type = other DNA; note = Primer
ccgcagtgatcataccttag

SEQ ID NO: 16 - DNA, 20 bp, synthetic construct
  source 1..20: mol_type = other DNA; note = Primer
tgtatggagtgaggaaggtt

SEQ ID NO: 17 - DNA, 20 bp, synthetic construct
  source 1..20: mol_type = other DNA; note = Primer
cagaatatcgcgggacgcag

SEQ ID NO: 18 - DNA, 25 bp, synthetic construct
  source 1..25: mol_type = other DNA; note = Primer
accaaggggtgatctctcgagatgg

SEQ ID NO: 19 - DNA, 22 bp, synthetic construct
  source 1..22: mol_type = other DNA; note = Primer
tgctcgtctgagacggcttggg

SEQ ID NO: 20 - DNA, 22 bp, synthetic construct
  source 1..22: mol_type = other DNA; note = Primer
gaggccgtggttggcttgtatg

SEQ ID NO: 21 - DNA, 20 bp, synthetic construct
  source 1..20: mol_type = other DNA; note = Primer
ccgggtatactagtgatttg

SEQ ID NO: 22 - DNA, 59 bp, synthetic construct
  source 1..59: mol_type = other DNA; note = Primer
tggatccgttgggattcaaatggtctcaaagtgaggttgaaccatgatggcagtgttcg

SEQ ID NO: 23 - DNA, 58 bp, synthetic construct
  source 1..58: mol_type = other DNA; note = Primer
ctgccatcatggttcaacctcactttgagaccatttgaatcccaacggatccacattg

SEQ ID NO: 24 - DNA, 59 bp, synthetic construct
  source 1..59: mol_type = other DNA; note = Primer
gggtcgtttgttcaaatcactagtatacccggataggttgatgatacacagcattctcg

SEQ ID NO: 25 - DNA, 57 bp, synthetic construct
  source 1..57: mol_type = other DNA; note = Primer
gccgagaatgctgtgtatcatcaacctatccgggtatactagtgatttgaacaaacg

SEQ ID NO: 26 - DNA, 60 bp, synthetic construct
  source 1..60: mol_type = other DNA; note = Primer
gctgagaatattgtaggagatcttctagaaagatgctgcagacaagttcctgcagctgtg

SEQ ID NO: 27 - DNA, 57 bp, synthetic construct
  source 1..57: mol_type = other DNA; note = Primer
ccagatcttccggatggctcgagtttttcagcaagtgctttattcaggcacgctacg

SEQ ID NO: 28 - DNA, 49 bp, synthetic construct
  source 1..49: mol_type = other DNA; note = Primer
tgcaggaacttgtctgcagcatctttctagaagatctcctacaatattc

SEQ ID NO: 29 - DNA, 59 bp, synthetic construct
  source 1..59: mol_type = other DNA; note = Primer
gaagaaaaggctgtcatgaatttagtttacgtgaggttgaaccatgatggcagtgttcg

SEQ ID NO: 30 - DNA, 59 bp, synthetic construct
  source 1..59: mol_type = other DNA; note = Primer
cgaacactgccatcatggttcaacctcacgtaaactaaattcatgacagccttttcttc

SEQ ID NO: 31 - DNA, 59 bp, synthetic construct
  source 1..59: mol_type = other DNA; note = Primer
gtcgtttgttcaaatcactagtatacccggtgaattctagaatgtgaggtggaatgagg

SEQ ID NO: 32 - DNA, 60 bp, synthetic construct
  source 1..60: mol_type = other DNA; note = Primer
atattctcacgtagcgtgcctgaataaagcacttgctgaaaaactcgagccatccggaag

SEQ ID NO: 33 - DNA, 55 bp, synthetic construct
  source 1..55: mol_type = other DNA; note = Primer
cttccggatggctcgagtttttcagcaagatcagacgcattggctgccttctcag

SEQ ID NO: 34 - DNA, 55 bp, synthetic construct
  source 1..55: mol_type = other DNA; note = Primer
cgggctgagaaggcagccaatgcgtctgatcttgctgaaaaactcgagccatccg

SEQ ID NO: 35 - DNA, 58 bp, synthetic construct
  source 1..58: mol_type = other DNA; note = Primer
cacaacacaacgatcggcagagcagtaatctttctagaagatctcctacaatattctc

SEQ ID NO: 36 - DNA, 56 bp, synthetic construct
  source 1..56: mol_type = other DNA; note = Primer
gaatattgtaggagatcttctagaaagattactgctctgccgatcgttgtgttgtg

SEQ ID NO: 37 - DNA, 60 bp, synthetic construct
  source 1..60: mol_type = other DNA; note = Primer
ctcgatggagtggtaagccactgccattgggtaaactaaattcatgacagccttttcttc

SEQ ID NO: 38 - DNA, 60 bp, synthetic construct
  source 1..60: mol_type = other DNA; note = Primer
aagaagaaaaggctgtcatgaatttagtttacccaatggcagtggcttaccactccatcg

SEQ ID NO: 39 - DNA, 53 bp, synthetic construct
  source 1..53: mol_type = other DNA; note = Primer
gcctcattccacctcacattctagaattcacttccatgggaggctaagaaacg

SEQ ID NO: 40 - DNA, 58 bp, synthetic construct
  source 1..58: mol_type = other DNA; note = Primer
aaacgcacgtttcttagcctcccatggaagtgaattctagaatgtgaggtggaatgag

SEQ ID NO: 41 - DNA, 48 bp, synthetic construct
  source 1..48: mol_type = other DNA; note = Primer
gcctcattccacctcacattctagaattcagtaaactaaattcatgac

SEQ ID NO: 42 - DNA, 40 bp, synthetic construct
  source 1..40: mol_type = other DNA; note = Primer
ggctgtcatgaatttagtttactgaattctagaatgtgag

SEQ ID NO: 43 - DNA, 20 bp, synthetic construct
  source 1..20: mol_type = other DNA; note = Primer
tgtcgtcggtcggtgagtag

SEQ ID NO: 44 - DNA, 19 bp, synthetic construct
  source 1..19: mol_type = other DNA; note = Primer
atagaatccaccggcgttg

SEQ ID NO: 45 - DNA, 53 bp, synthetic construct
  source 1..53: mol_type = other DNA; note = Primer
atgataggagttttaacaacgccggtggattctatggccgcctgcaggtcgac

SEQ ID NO: 46 - DNA, 52 bp, synthetic construct
  source 1..52: mol_type = other DNA; note = Primer
ttgggagagcgactactcaccgaccgacgacagcccaattcgccctatagtg

SEQ ID NO: 47 - DNA, 48 bp, synthetic construct
  source 1..48: mol_type = other DNA; note = Primer
ccgaactcgttaaggtcaatccactcagccaacactgcaacgcatttg

SEQ ID NO: 48 - DNA, 40 bp, synthetic construct
  source 1..40: mol_type = other DNA; note = Primer
cgttgcagtgttggctgagtggattgaccttaacgagttc

SEQ ID NO: 49 - DNA, 53 bp, synthetic construct
  source 1..53: mol_type = other DNA; note = Primer
gatcagaattcgaacactgccatcatggttcaacgattgaccttaacgagttc

SEQ ID NO: 50 - DNA, 55 bp, synthetic construct
  source 1..55: mol_type = other DNA; note = Primer
gttgggtcgtttgttcaaatcactagtatacccggcactcagccaacactgcaac

SEQ ID NO: 51 - DNA, 19 bp, synthetic construct
  source 1..19: mol_type = other DNA; note = Primer
cagaacctgcaaggagaac

SEQ ID NO: 52 - DNA, 19 bp, synthetic construct
  source 1..19: mol_type = other DNA; note = Primer
acggacgttgcgttcatgc

SEQ ID NO: 53 - DNA, 20 bp, synthetic construct
  source 1..20: mol_type = other DNA; note = Primer
tccataggtctcacccattc

SEQ ID NO: 54 - DNA, 19 bp, synthetic construct
  source 1..19: mol_type = other DNA; note = Primer
accgggttagctctgttgc

SEQ ID NO: 55 - DNA, 54 bp, synthetic construct
  source 1..54: mol_type = other DNA; note = Primer
ccgatgaggaaggagaatgggtgagacctatggagcccaattcgccctatagtg

SEQ ID NO: 56 - DNA, 55 bp, synthetic construct
  source 1..55: mol_type = other DNA; note = Primer
gatcagaattcgaacactgccatcatggttcaacgaattccgcttagtctcgtac

SEQ ID NO: 57 - DNA, 19 bp, synthetic construct
  source 1..19: mol_type = other DNA; note = Primer
ctagcgctgccaatgtaac

SEQ ID NO: 58 - DNA, 50 bp, synthetic construct
  source 1..50: mol_type = other DNA; note = Primer
ggtcgtttgttcaaatcactagtatacccggcagctctgtggcgaaatac

SEQ ID NO: 59 - DNA, 19 bp, synthetic construct
  source 1..19: mol_type = other DNA; note = Primer
attctggggaggatgaatc

SEQ ID NO: 60 - DNA, 48 bp, synthetic construct
  source 1..48: mol_type = other DNA; note = Primer
ctgccgcagaggattcatcctccccagaatggccgcctgcaggtcgac

SEQ ID NO: 61 - DNA, 1299 bp, Starmerella bombicola
  source 1..1299: mol_type = genomic DNA
atggccatcgagaaaccagtgatagttgcttgtgcctgcccactagcggggcacgtgggcccagtgctcagcctggtccgcggtctactcaatagaggatatgaggtgactttcgtaacagggaacgcattcaaggagaaagttattgaggcaggatgcactttcgtccctctccaaggacgagctgactaccatgaatacaatctccctgaaatcgctccaggattgctcacgattcctccaggccttgagcagaccggttactcaatgaatgagatttttgtgaaggcgattcctgagcagtacgatgcacttcaaactgctctaaaacaggttgaggctgaaaataaatcagctgtggtgattggcgagaccatgtttctaggggtgcatccgatatcactgggtgccccaggtctcaagccccaaggcgtaatcacgttaggaactattccgtgcatgctgaaagcagagaaggcgcctggagttcctagtcttgagccaatgattgatactttagtgcggcaacaagtatttcaaccaggaactgactctgagaaggagatcatgaagacgctcggggccacgaaggagcccgaatttctcctggagaatatatacagcagccctgacagatttttgcaactgtgccctccatctcttgaatttcacttgacttcgcctcctcctggcttctcgttcgctggtagtgcaccgcatgtaaagtctgctggattagcaactccacctcacctgccgtcttggtggcctgatgtgctgagtgcgaagcgtctgattgttgttacacaaggaacagcagccatcaactatgaagatctgctcattccagcattgcaggcctttgctgacgaagaagacactctcgtagttggtatattgggcgtcaaaggggcgtcacttcctgatagcgttaaagttcctgcaaacgctcgaattgttgattattttccttacgatgagctactaccgcatgcctctgttttcatatacaacggtggatacggaggtctgcagcacagtttgagccatggcgttcccgtcatcatcggaggaggaatgttggtagacaagccagctgttgcttcacgagctgtatgggctggtgttggttatgatcttcaaaccttgcaggcaacttctgagctagtctccacggccgttaaggaggtgttggctactccctcgtatcacgagaaagccatggcagtcaagaaagagcttgaaaaatacaagtctcttgatattctagagtcggcaattagtgaattagcttcttaa

SEQ ID NO: 62 - protein, 432 aa, Starmerella bombicola
MAIEKPVIVACACPLAGHVGPVLSLVRGLLNRGYEVTFVTGNAFKEKVIEAGCTFVPLQGRADYHEYNLPEIAPGLLTIPPGLEQTGYSMNEIFVKAIPEQYDALQTALKQVEAENKSAVVIGETMFLGVHPISLGAPGLKPQGVITLGTIPCMLKAEKAPGVPSLEPMIDTLVRQQVFQPGTDSEKEIMKTLGATKEPEFLLENIYSSPDRFLQLCPPSLEFHLTSPPPGFSFAGSAPHVKSAGLATPPHLPSWWPDVLSAKRLIVVTQGTAAINYEDLLIPALQAFADEEDTLVVGILGVKGASLPDSVKVPANARIVDYFPYDELLPHASVFIYNGGYGGLQHSLSHGVPVIIGGGMLVDKPAVASRAVWAGVGYDLQTLQATSELVSTAVKEVLATPSYHEKAMAVKKELEKYKSLDILESAISELAS